(19) United States
(12) Patent Application Publication
(10) Pub. No.: US 2007/0280989 A1
(43) Pub. Date: Dec. 6, 2007

(54) Compositions and Methods for Stem Cell Expansion and Differentiation

(75) Inventors: Abraham Shahar, Rehovot (IL); Zvi Nevo, Herzliya (IL); Shimon Rochkind, Ramat Aviv Gimel (IL)

Correspondence Address: FENNEMORE CRAIG, 3003 NORTH CENTRAL AVENUE, SUITE 2600, PHOENIX, AZ, 85012, US

(73) Assignee: NVR LABS LTD, Ness Ziona (IL)

(21) Appl. No.: 11/575,618

(22) PCT Filed: Sep. 21, 2005
(86) PCT No.: PCT/IL05/01007

§ 371 Date: May 22, 2007

Related U.S. Application Data
(60) Provisional application No. 60/611,566, filed on Sep. 21, 2004.
(60) Provisional application No. 60/706,014, filed on Aug. 8, 2005.

(51) Int. Cl. C12N 5/06; A61K 35/12; A61K 9/00; C12N 5/10; A61P 41/00; C12N 15/86

(52) U.S. Cl. 424/423; 424/93.21; 424/93.7; 435/377; 435/395; 435/397; 435/456

(57) ABSTRACT

The present invention relates to compositions comprising stem cells and partially committed progenitor cells and to methods of controlling cell proliferation and differentiation, which can be used for expansion of stem cells and their subsequent differentiation. The present invention provides expanded population of essentially undifferentiated stem cells, which are useful in clinical procedures involving stem cell therapy, and a population derived thereof of which at least part of the cells are differentiated. The cells can be used per se, as a part of a cell-bearing composition comprising cross-linked hyaluronic acid-laminin gels or as a part of a composite implant for tissue regeneration.

### FIELD OF THE INVENTION

The present invention relates to compositions and methods for obtaining expanded stem cells, specifically to compositions comprising expanded stem cells and partially committed progenitor cells, to use thereof for directed differentiation, and as a component of composite implants, and to the use of the composite implants for transplantation and tissue regeneration.

### BACKGROUND OF THE INVENTION

Stem cells are primitive undifferentiated cells having the capacity to mature into other cell types, for example, brain, muscle, liver and blood cells. Stem cells are typically classified as either embryonic stem cells, or adult tissue derived-stem cells, depending on the source of the tissue from which they are derived. Pluripotent stem cells are undifferentiated cells having the potential to differentiate to derivatives of all three embryonic germ layers (endoderm, mesoderm, and ectoderm). Adult progenitor cells are adult stem cells which can give rise to a limited number of particular types of cells.

Pluripotent human embryonic stem cells provide biomedical research with new approaches for drug development and testing, and for organ repair and replacement. Unlike all current treatments relying upon surgical intervention or drugs that modulate physiological activities, stem cells provide a replacement for dysfunctional or degenerating tissue.

Replacement therapy using stem cells could dramatically change the prognosis of many untreatable diseases. For example, many neurological diseases, such as disorders of the brain, spinal cord, peripheral nerves and muscles, are characterized by the sudden or gradual death of brain or muscle cells. These diseases which include stroke, head and spinal cord trauma, Alzheimer's Disease, Parkinson's Disease, Multiple sclerosis, Amyotrophic lateral sclerosis (ALS), genetic enzyme deficiencies such as Gaucher disease, Muscular dystrophy and others could potentially be treated using stem cell replacement therapy.

Use of primordial cells from human embryos for implantation therapy in spinal cord injuries is gaining more and more attention. However, despite their significant therapeutic potential, stem cells are not widely used in cell replacement and tissue regeneration therapies. This is partially due to their low availability and their limited capacity for expansion in common ex vivo culturing methods.

The most common method for culturing embryonic stem (ES) cells is based on mouse embryonic fibroblasts (MEF) as a feeder cell layer supplemented with tissue culture medium containing serum or leukemia inhibitor factor (LIF) which supports the proliferation and the pluripotency of the ES cells (Thomson et al., 1998. Science 282:1145-1147). MEF cells are derived from day 12-13 mouse embryos in a medium supplemented with fetal bovine serum. Under these conditions ES cells can be maintained for many passages in culture while preserving their phenotypic and functional characteristics. However, unlike mouse ES cells, the presence of added LIF does not prevent differentiation of the human ES cells. Furthermore, the use of feeder cells substantially increases the cost of production, and makes scale-up of human ES cell culture impractical. Additionally, the feeder cells are inactivated to arrest their proliferation and to keep them from outgrowing the stem cells; hence it is necessary to have fresh feeder cells for each splitting of the human ES culture. Procedures are not yet developed for completely separating feeder cell components away from embryonic cells prepared in bulk culture. Thus, the presence of xenogeneic components from the feeder cells complicates their potential use in human therapy. Furthermore, feeder cells, whether allogeneic or xenogeneic, may introduce pathogens.

ES cells can also be cultured on MEF under serum-free conditions using serum replacements supplemented with basic fibroblast growth factor (bFGF) (Amit et al., 2000. Dev. Biol. 227:271-278). Under these conditions the cloning efficiency of ES cells is four times higher than with fetal bovine serum. In addition, following 6 months of culturing under serum replacement the ES cells still maintain their pluripotency as indicated by their ability to form teratomas which contain all three embryonic germ layers. Although this system uses better-defined culture conditions, the presence of mouse cells in the culture exposes the human culture to pathogens which restricts their use in cell-based therapy.

Pluripotent stem cells can be obtained from various sources. Embryonic stem cells can be isolated or propagated from blastocysts of human or other mammalian source. Established human embryonic stem cell lines and their equivalents are also available. Other commonly used sources for stem cells include cells isolated from umbilical cord blood and cells isolated from other tissues or germ layers comprising stem cells. The recent discoveries that hematopoietic stem cells can give rise to non-hematopoietic tissues suggest that these cells may have greater differentiation potential than was previously assumed and open new frontiers for their therapeutic applications (Krause, D. S. et al., 2001. Cell 105:369-377). Studies have shown that cord blood-derived stem cells are capable of repairing neurological damage caused by brain injuries and strokes and are also capable of functional and morphological incorporation into animal heart tissue.

US Patent Application No. 20040067580 discloses an animal-free culturing system for stem cells comprising human foreskin cells capable of maintaining stem cells in an undifferentiated state when co-cultured therewith.

US Patent Application No. 20030017589 discloses a culture environment containing an extracellular matrix made from isolated extracellular matrix components such as Matrigel and laminin that supports proliferation of human embryonic stem cells wherein the role of feeder cells is replaced by components added to the culture environment that support rapid proliferation without differentiation.

US Patent Application No. 20020137204 discloses a system for culturing human pluripotent stem (pPS) cells in the absence of feeder cells wherein the feeder cells are replaced by supporting the culture on an extracellular matrix such as Matrigel, laminin, or collagen. However, the disclosed method still requires culturing the cells in a conditioned medium, produced by permanent cell lines.

Another potential complication in using human embryonic stem cells for replacement and tissue regeneration therapies is that the cells would be considered as an allogeneic graft, and should overcome the risks of rejection, immunogenic reaction and possible neoplastic transformation. Adult stem cells, including partially committed progenitor cells, can answer this limitation.

Spinal cord injuries involving partial or complete transection, as with other lesions in the central nervous system, are unable to heal on their own. Complete spinal cord injuries in humans and other mammals cause loss of sensory, motor and reflex functions below the site of injury. Nerve regeneration is largely considered an unattainable goal within the central nerve system (CNS), due to the inability of these cell types to multiply after maturation of the brain, which occurs early in life. Axonal injury within the central nervous system is also generally thought to be irreversible.

Several different approaches have been used in attempting to reconstruct an injured spinal cord. The use of growth factors, either by exogenous administration or by introducing growth factor-treated implants and genetically engineered cells has been attempted with limited success. Others have concentrated their efforts on the use of various tissue-engineered scaffolds. Spinal cord reconstruction using implantation of cells from various sources has been also studied in recent years. However, one of the major disadvantages of the implantation or injection of cells alone is the limited viable cell survival after the procedure, as cells tend to desert the injury site.

An excellent autologous source of adult neuronal precursor cells is the nasal olfactory mucosa (NOM) (Veyrac et al., 2005, Eur J Neurosci. 21(10): 2635-2648). The NOM tissue comprises an epithelial cell layer containing sustentacular supporting cells, basal cells, immature neurons, mature sensory neurons and lamina propria containing ensheathing, glial cells, endothelial cells, fibroblasts and glandular cells. The NOM tissue is easily biopsied and the neurons and the sustentacular cells of the NOM mucosa renew themselves constantly during life by proliferating of the basal global stem cells. Olfactory ensheathing cells enwrap axons of olfactory nerves in olfactory nerve bundles in the lamina propria and in the olfactory bulb; the olfactory bulb is the site of olfactory nerve axon termination in the brain. The olfactory ensheathing cells are specialized glia, which have two interesting and useful properties. Like Schwann cells of the peripheral nervous system, ensheathing cells permit and promote axon growth, properties not seen in the glia of the central nervous system. However, unlike Schwann cells, olfactory ensheathing cells exist both within and outside the central nervous system.

WO 01/30982 discloses a method of isolating ensheathing cells, preferably from isolated olfactory lamina propria, and use of the isolated ensheathing cells or isolated lamina propria in transplantation, particularly transplantations directed to neural regions (for example brain, spine and/or peripheral nerves) of a human to assist recovery of acute and chronic nerve damage following surgery or trauma.

Transplantation of ensheathing cells from the olfactory nerve layer of the olfactory bulb has been recently shown to be successful in achieving functional recovery after adult spinal cord lesions (See, for example the review of Lu J. and Waite P. 1999. Spine 24:926-920).

However, implantation or injection of cells alone has major disadvantages such as limited cell viability after the procedure and cells deserting the injury site.

An alternative way of repairing injured mammalian spinal cord may therefore be by creating a composite implant, which contains cultured cells from autologous or allogeneic source. The attributes of an ideal biocompatible implant would include the ability to support cell growth either in-vitro or in-vivo, specifically the ability to support growth and differentiation of the desired cell types, the ability to anchor the implanted cell to the site of injury while still having the desired degree of flexibility, the ability to have varying degrees of biodegradability, the ability to be introduced into the intended site in vivo without provoking secondary damage, and the ability to serve as a vehicle or reservoir for delivery of drugs or bioactive substances to the desired site of action.

WO 02/39948 to some of the inventors of the present invention discloses a biocompatible combined gel comprising hyaluronic acid and laminin cross-linked by an exogenous cross-linking agent (defined as HA-LN-Gel). The HA-LN-Gel affords a convenient environment for cell attachment, growth, differentiation and tissue repair, and it may be used either in vitro or in vivo. The laminin component stabilizes the cells, provides cell attachment sites and improves cell viability, particularly of cells that are intended for use in tissue regeneration. However, as laminin on its own suffers from the drawback that its physical characteristics are inappropriate for use in an implant, the gel further comprises the hyaluronic acid component that provides the physical attributes required to enable the laminin to fulfill its purpose. The combined laminin and HA gels are further stabilized by cross-linking, to provide the gel with the desired degree of biodegradability, porosity and elasticity.

WO 2004/029095 to some of the inventors of the present invention discloses cohesive biopolymers comprising a coprecipitate of a sulfated polysaccharide and a fibrillar protein, specifically a coprecipitate of dextran sulfate and gelatin. The cohesive biopolymer is biocompatible and is useful as a scaffold for cell free or cell bearing implants for use in vitro or in vivo.

There is an ongoing need for culturing systems capable of supporting embryonic stem cell proliferation in culture for extended periods of time. Thus, it would be highly advantageous to have a new composition for growing, expanding and manipulating stem cells without the need of a feeder layer, together with means for successful cell implantation and tissue regeneration, including biocompatible implants.

### SUMMARY OF THE INVENTION

The present invention relates to compositions and methods for expanding pluripotent stem cells and partially committed progenitor cells, wherein the expanded cells can further undergo differentiation, and use thereof, particularly as a component of a composite implant for tissue regeneration.

The present invention relates to compositions and methods for expanding stem and progenitor cells in their undifferentiated state. The present invention provides a system comprising expanded stem cells, including pluripotent embryonic stem cells and partially committed stem cells, cultured in or on biocompatible matrices comprising cross-linked hyaluronic acid-laminin gels (HA-LN-Gels), wherein the majority of the cells remain undifferentiated.

Unexpectedly, the system is devoid of a feeder layer and yet successfully supports extended ES cell growth and proliferation. Thus, the composition of the present invention provides high quality expanded stem and progenitor cells, which are not contaminated by any debris or component of other cell or tissue types and can be used in human therapy. Furthermore, the compositions comprising the expanded stem cells are also suitable for differentiating the cell to a desired cell type.

The present invention further relates to composite implants comprising cells cultured in or on cross-linked hyaluronic acid-laminin gels, further comprising a scaffold, and to the use of the composite implant for tissue regeneration, specifically for neuronal regeneration and treatment of spinal cord injury. The cells cultured in or on HA-LN-Gel comprise at least one type of pluripotent stem cells, partially committed progenitor cells, differentiated cells or a combination thereof.

The present invention is based in part on the discovery that stem cells, either pluripotent cells or partially committed progenitor cells cultured in or on HA-LN-Gel in appropriate culture media proliferate in the culture and maintain an undifferentiated state. Particularly, the present invention discloses that embryonic stem cells and neuronal precursor cells from biopsies of adult nasal olfactory mucosa (NOM) cultured in or on HA-LN-Gel can maintain their substantially undifferentiated state. The present invention further discloses that the embryonic stem cells and expanded NOM can progress into a differentiated state and can be further cultured in or on the HA-LN-Gel either in vitro under appropriate conditions or in vivo after implantation into a mammalian body.

According to one aspect, the present invention provides a composition for expanding stem cells, comprising a population of stem cells cultured in or on a biocompatible matrix comprising hyaluronic acid and laminin cross-linked to form a combined gel, wherein at least the majority of the cells maintain their undifferentiated state.

According to one embodiment, the composition for expanding stem cells is devoid of a feeder layer. According to preferred embodiments, the composition for expanding stem cells is devoid of either a feeder layer or any conditioned medium.

According to certain embodiments, the stem cells are of human origin. According to additional embodiments, the stem cells are of non-human mammalian origin. According to one embodiment, the stem cells are selected from embryonic stem cells and adult stem cells. The adult stem cells can be pluripotent or partially committed progenitor cells. According to certain embodiments, the cells proliferate in the culture. In specific embodiments, at least some of the cells form a monolayer in the cell culture. According to other embodiments, at least some of the cells form an embryoid body structure in the cell culture. According to yet other embodiments, the cells maintain their undifferentiated state through at least one passage of the cell culture, preferably through a plurality of passages.

According to one embodiment, the composition comprises genetically modified stem cells. Typically, the cells are transformed with a suitable vector comprising an exogene for effecting the desired genetic alteration, as is known to a person skilled in the art.

The composition of the present invention may comprise stem cells of various types, for example stem cells isolated or propagated from blastocysts of human or other mammalian source, including established human embryonic stem cell lines and their equivalents; stem cells isolated from umbilical cord blood; and stem cells isolated from other tissues or germ layers comprising stem cells.

According to one embodiment, the stem cells may be partially committed progenitors isolated from several tissue sources, selected from the group consisting of hematopoietic cells, neural progenitor cells, oligodendrocyte cells, skin cells, hepatic cells, muscle cells, bone cells, mesenchymal cells, pancreatic cells, chondrocytes and marrow stromal cells. According to certain currently preferred embodiments of the present invention, neural progenitor cells are obtained from nasal olfactory mucosa (NOM).

The composition may contain a homogenous cell population or a mixed population of cell types or cell lines. According to one embodiment, the expanded undifferentiated culture is derived from a single type of stem cells, and thus comprises cells having the same genotype. According to another embodiment, the culture comprises mixed populations made by combining different lines of stem cells and their progeny. According to one embodiment, the stem cells are enriched for a specific cell type including, but not limited to, CD34⁺, CD34-depleted cell population, CD133⁺ cells, CD133-depleted cell populations and combinations thereof. According to yet another embodiment, the expanded undifferentiated culture is derived from a tissue comprising a plurality of cell types, including stem cells and partially committed progenitors cells.

According to certain currently preferred embodiments, the partially committed progenitor cells are nasal olfactory mucosa (NOM) cells. The NOM is currently considered as the only cell source potentially available for adult human autologous neuronal precursor cells.

The present inventions discloses that embryonic stem cells and neuronal progenitor cells isolated from the NOM cultured in or on HA-LN-Gel can maintain their undifferentiated state and expand in vitro without the need of a feeder layer, while maintaining their ability to differentiate either in vitro in appropriate culture media or in vivo when the composition is implanted into a mammalian body. The composition of the present invention is advantageous over previously known compositions as it can be used for both undifferentiated cell expansion to reach sufficient amount of cells, and subsequent differentiation of the cells either in vitro or in vivo after implantation within the body.

The cell culture typically includes a culture medium comprising an isotonic buffer, a protein or amino acid source, nucleotides, lipids, and optionally hormones and the like. According to one embodiment, the culture medium is serum free. According to another embodiment, the culture medium comprises serum. Typically, serum-containing culture media are used for expansion of undifferentiated cells in or on a HA-LN-Gel, while serum-free culture media are used for inducing differentiation of the cells in or on the HA-LN-Gel. According to certain currently preferred embodiments, the serum is an autologous serum. According to other embodiments, the serum is a non-autologous serum.

According to another embodiment, the culture medium is enriched with an agent which supports the growth of the cells in an undifferentiated state. Appropriate agents which support the growth of the cells in an undifferentiated state include but are not limited to growth factors such as basic fibroblast growth factor (bFGF), epidermal growth factor (EGF), members of the interleukin 6 (IL-6) family and leukemia inhibitory factor (LIF). According to certain currently preferred embodiments expansion of undifferentiated NOM cells is obtained with a medium comprising serum without additional factors, and expansion of embryonic stem cells is obtained with a medium comprising serum, LIF and bFGF. It is to be understood that the composition of the present invention does not require the presence of a conditioned medium, as the hyaluronic acid-laminin milieu supplemented with nutrient medium and appropriate growth factors is sufficient to keep the stem cells in a substantially undifferentiated state throughout the expansion of the culture.

According to yet another embodiment, the culture medium is enriched with an agent which supports the differentiation of the stem cell in or on the HA-LN-Gel. According to one embodiment, the agents supporting differentiation are selected from the group consisting of, but not limited to growth factors, neurotransmitors, and small molecules serving as growth and differentiation regulators.

According to certain embodiments of the present invention, the expanded cell culture is used as a medical implant. In some embodiments, differentiation of the cells is required prior to implantation. Accordingly, appropriate agents, which support the growth and differentiation of the cells, are added to the culture medium.

According to certain currently preferred embodiments, the expanded cells used for implantation are NOM or embryonic spinal cord cells. According to additional embodiments, expansion and differentiation of NOM cells is supported by a growth factor selected from the group consisting of brain-derived neurotrophic factor (BDNF); bFGF, nerve growth factor (NGF), dopamine, retinoic acid EGF or a combination thereof.

The HA-LN-Gel is described in WO 02/39948 to some of the inventors of the present invention, incorporated in its entirety by reference as if fully set forth herein. The transparent HA-LN-Gel affords a convenient environment for cell attachment and growth. Furthermore, the HA-LN-Gel provides a hydrophilic environment and facilitates sustained release of bioactive components. Advantageously, during the production of HA-LN-Gel compositions it is possible to control the viscosity and the degree of elasticity or malleability of the composition, as well as other properties including biodegradability, porosity (which contribute to the rate in which substances can diffuse from the gel), and other attributes.

According to additional embodiments, it is possible to include synthetic or natural polymers in the form of a plurality of carriers dispersed within the gel. According to certain embodiments, the carriers are microcarriers. According to certain currently preferred embodiments, the microcarriers are positively charged.

According to another aspect, the present invention provides a composite implant comprising cells cultured in or on HA-LN-Gel, further comprising a biocompatible scaffold. In certain embodiments, the biocompatible scaffold encloses the cells cultured in or on the gel.

According to some embodiments, the composite comprises cells selected from undifferentiated stem cells, differentiated cells or a combination thereof. According to one embodiment, the stem cells are selected from the group consisting of pluripotent embryonic stem cells and partially committed progenitors cells. According to certain currently preferred embodiments, the partially committed progenitor cells are NOM cells. According to another embodiment, the cells are differentiated cells. According to certain currently preferred embodiment, the cells are neural cells. According to additional currently preferred embodiments, the neural cells are selected from embryonic spinal cord neuronal cells and neuronal precursor cells differentiated from partially committed NOM cells. According to one embodiment, the cells are of human origin. According to another embodiment, the cells are of nonhuman mammalian origin.

The biocompatible scaffold may comprise any appropriate material known in the art. According to certain embodiments, the biocompatible scaffold comprises a cohesive biopolymer comprising a coprecipitate of at least one fibrillar protein and at least one sulfated polysaccharide as described in WO 2004/029095 to some of the inventors of the present invention, incorporated herein in its entirety by reference. According to certain currently preferred embodiments, the scaffold is a coprecipitate of dextran sulfate and gelatin. As described in WO 2004/029095 the scaffold can be shaped to various forms as a support for cell culture. According to certain embodiments, the scaffold is shaped to a tubular form, specifically tubular grooved form. According to additional embodiments, the tubular scaffold contains nanofibers made of the same material as the scaffold. According to certain currently preferred embodiments, the nanofibers are in a shape of a bundle of parallel nanofibers. The scaffold is non-toxic and non-inflammatory, and its attributes, including, for example, elasticity, rigidity and biodegradability can be controlled during production. According to certain embodiments, the scaffold is positively charged. According to certain currently preferred embodiments, the scaffold may be sutured without damage to the overall structure.

According to a further aspect, the present invention provides a method for expanding stem cells, comprising: (a) providing a population of stem cells; and (b) culturing the population of stem cells in or on a composition comprising biocompatible matrix comprising hyaluronic acid and laminin cross-linked to form a combined gel; wherein the cultured cells are proliferating while substantially maintaining their undifferentiated state.

According to a preferred embodiment, the composition used in these methods for expanding stem cells is devoid of either a feeder layer or any conditioned medium.

According to certain embodiments, the population of the stem cells is obtained from a pre-culture grown on a feeder layer in a serum containing or serum free culture medium. Alternatively, isolated stem cells are directly seeded in or on the HA-LN-Gel.

According to one embodiment, the method for expanding stem cells utilizes a population of genetically modified stem cells. According to another embodiment, the method of expanding stem cells further comprises a step of transforming the population of the stem cell with suitable vector comprising an exogene. The transformation step may be performed before culturing the stem cell population in or on the HA-LN composite gel, or before re-seeding isolated cells during culture passages.

Many vectors suitable for use in cellular gene therapy are known in the art. The vector can be, for example, a plasmid, a bacteriophage, a virus or a non-viral transformation system such as a nucleic acid/liposome complex. Similarly, a range of nucleic acid vectors can be used to genetically transform the expanded cells of the invention. Alternatively, the nucleic acid encoding the gene product (including the necessary regulatory elements) is contained within a plasmid vector.

According to yet another aspect, the present invention provide a method for differentiating stem cells, comprising: (a) providing a population of stem cells; and (b) culturing the population of stem cells in or on a composition comprising biocompatible matrix comprising hyaluronic acid and laminin cross-linked to form a combined gel; wherein at least part of the stem cells differentiate to a desired cell type.

According to certain embodiments, the method further comprises culturing the cells in a suspension comprising microcarriers (Mc) before culturing in or on the HA-LN-Gel. According to additional embodiments, cells are cultured alternately as stationary cultures in or on the HA-LN-Gel and subsequently in suspension on microcarriers, with a final stationary growth in or on the HA-LN-Gel. According to one currently preferred embodiment, the microcarriers are positively charged, to enable cell attachment in order to form floating cell-Mc aggregates.

According to certain embodiments, the stem cells are selected from pluripotent stem cells, partially committed progenitor cells or a combination thereof. According to certain embodiments, the progenitor cells are neural progenitor cells. According to certain currently preferred embodiments, the neural progenitor cells are NOM cells.

According to yet another aspect, the present invention provides a method for transplanting cells to an individual in need thereof, comprising the step of transplanting a composite implant comprising cells cultured in or on the HA-LN-Gel, further comprising a biocompatible scaffold, wherein the scaffold supports the cell culture. According to certain embodiments, the cells are transplanted into a site of an injured tissue. According to certain currently preferred embodiments, the cells are transplanted into an injured site of spinal cord tissue. According to one embodiment, the method further comprises covering the site of an injured tissue with a thin biodegradable membrane for fixation of the implants at the injured site. According to one embodiment, the membrane comprises a coprecipitate of dextran sulfate and gelatin. According to another embodiment, the membrane is attached to the injured site by interstitial sutures.

According to certain embodiments, the cells are selected from undifferentiated stem cells, differentiated cells or a combination thereof. According to one embodiment, the stem cells are selected from the group consisting of pluripotent embryonic stem cells and partially committed progenitors cells. According to certain currently preferred embodiments, the cells are partially committed NOM cells. According to another embodiment, the cells are differentiated cells. According to certain currently preferred embodiment, the cells are neural cells. According to additional currently preferred embodiments, the neural cells are selected from neural embryonic spinal cord cells and neuronal precursor cells differentiated from partially committed NOM cells. According to one embodiment, the cells are of autologous source. According to another embodiment, the cells are of allogeneic source.

These and other embodiments of the present invention will become apparent in conjunction with the figures, description and claims that follow.

### BRIEF DESCRIPTION OF THE FIGURES

FIG. 1 shows micrographs of human embryonic stem cells (hES) grown in HA-LN-Gel. Section A displays hES cell-aggregates soon after embedding in HA-LN-Gel. Section B shows a micrograph of the hES 15 hours after embedding in the HA-LN-Gel. Sections C-E show micrographs of the hES 8, 22 and 24 days after embedding in the HA-LN-Gel. Section F shows a micrograph of the hES 24 days after embedding in the HA-LN-Gel. In Sections C-F, formed monolayers are visible, showing cells which differ in size and shape. Original magnification: sections A, D-F-200×, sections B&C-400×.

FIG. 2 shows micrographs of three-dimensional growth of hES cells grown in HA-LN-Gel for 22 days. Original magnification: section A-200×, section B-100×.

FIG. 3 shows a micrograph of undifferentiating bovine blastocyte cells grown in HA-LN-Gel. Inner cell mass (ICM) of bovine blastocyte (white arrow) inside the remains of the zona pelicuda (black arrow) 1 day after seeding in HA-LN-Gel. Original magnification: 400× (section A). Cellular growth and migration from the ICM, two weeks after seeding in HA-LN-Gel. Original magnification: 200× (section B). Growth of undifferentiated cells 4 days after enzymatic dissociation and re-seeding in HA-LN-Gel. Original magnification: 200× (section C).

FIG. 4 shows a micrograph of aggregates of undifferentiating dividing human umbilical blood cells grown for 8 days in HA-LN-Gel.

FIG. 5 shows a tubular scaffold containing nano-fibers. Original magnification ×25.

FIG. 6 shows phase contrast microscopy of mature motor neuron (section A) and myelinated axons (section B) (arrows) in long-term cultures of human embryonic spinal cord cells. Original magnification ×400.

FIG. 7 shows cultured adult human NOM neurons. Sections A-D show sprouting of nerve fibers concomitantly with migration of nerve cells from Mc-cell aggregates in HA-LN-Gel. Sections A-C: original magnification ×200; section D: original magnification ×100. Sections E and F show immunofluorescent staining of NOM neurons with antibodies specific to MAP 2 (section E) and olfactory mucosa protein (OMP, section F). Original magnification: section E: ×400, section F: ×200, respectively.

FIG. 8 shows rats after surgical treatment. Section A shows a complete paralysis of both legs, folded inward, of a control rat that underwent complete transection of the spinal cord and removal of a 4 mm segment. Section B shows paraplegic rat showing restoration of partial gait performance (in the right leg) three weeks after implantation of a composite implant containing cultured adult human NOM cells into a 4 mm gap of transected spinal cord.

FIG. 9 demonstrates an absence of spinal cord conductivity (SSEP) in a paraplegic control rat after complete transection of the spinal cord and removal of 4 mm segment (section A) and restoration of spinal cord conductivity after complete transection and implantation of composite implant containing NOM (section B).

FIG. 10 shows q-Space Displacement maps obtained by analyzing sequential axial slices of three different spinal cords accumulated by MRI.

FIG. 11 shows histological sections of implanted spinal cords ten months (sections A-C) after adult NOM implantation and three months (section D) after implantation of human embryonic spinal cord cells. Hematoxylin-Eosin (H&E) staining demonstrates dispersed neuronal perikarya (section A, arrows). Silver staining demonstrates nerve fibers either single (section B, arrows), or organized in parallel bundles (section D, arrows). In addition, note areas of neurokeratin (section. C, arrows). Original magnification ×400.

### DETAILED DESCRIPTION OF THE INVENTION

The present invention relates to compositions and methods of controlling proliferation and differentiation of pluripotent and partially committed stem cells, which can be used for expansion of stem cells and their subsequent differentiation. The compositions and methods of present invention can be used to provide expanded population of essentially undifferentiated stem cells, which are useful in clinical procedures involving stem cell therapy, and a population derived thereof of which at least part of the cells are differentiated. The cells can be used per se, as a part of a cell-bearing composition comprising cross-linked hyaluronic acid-laminin (HA-LN) gels or as a part of a composite implant for tissue regeneration.

Definitions

Stem cells are undifferentiated cells, which can give rise to a succession of mature functional cells. Embryonic stem (ES) cells are pluripotent, thus possessing the capability of developing into any organ or tissue type or, at least potentially, into a complete embryo. Adult stem cells are stem cells derived from tissues, organs or blood of an adult organism. The term embryonic-like stem cells refer to cells derived from tissues, organs or blood, possessing pluripotent characteristics of embryonic stem cells.

As used herein, the term “pluripotent stem cells” refers to cells that are: (i) capable of indefinite proliferation in vitro in an undifferentiated state; (ii) maintain a normal karyotype through prolonged culture; and (iii) maintain the potential to differentiate to derivatives of all three embryonic germ layers (endoderm, mesoderm, and ectoderm) even after prolonged culture.

The term “multipotent cells” known also as “multipotent adult progenitor cells (MAPCs)” or “partially committed progenitor cells” refers to adult stem cells which can give rise to a limited number of particular types of cells. For example, hematopoietic stem cells in the bone marrow are multipotent and give rise to the various types of blood cells.

As used herein, the term “nasal olfactory mucosa (NOM) cells” refers to cells obtained from the NOM tissue, typically by biopsy, and comprises a plurality of cell types. The NOM cells employed according to the teaching of the present invention can be either from autologous or allogeneic sources.

The term “undifferentiated” or “substantially undifferentiated” stem cells is used when a substantial proportion of stem cells and their derivatives in the population display morphological characteristics of undifferentiated cells, clearly distinguishing them from differentiated cells of embryo or adult origin. Undifferentiated stem cells are easily recognized by those skilled in the art, and typically appear in the two dimensions of a microscopic view with high nuclear/cytoplasmic ratios and prominent nucleoli. It is understood that colonies of undifferentiated cells within the population will often be surrounded by neighboring cells that are differentiated. Nevertheless, the undifferentiated colonies persist when the population is cultured or passaged under appropriate conditions, and individual undifferentiated cells constitute a substantial proportion of the cell population. Cultures that are substantially undifferentiated contain at least 20% undifferentiated stem cells, and may contain at least 40%, 60%, or 80% in order of increasing preference (in terms percentage of cells with the same genotype that are undifferentiated). The term “differentiated cells” refers to cells displaying the morphological characteristics of a certain cell type, as is known in the art. Neural cells are typically characterized by morphological observations (phase-construct microscopy) of bipolar or multipolar cells that cease proliferation, and by specific immunocytochemical staining. Cultures in which at least part of the cells are differentiated comprise at least 10% of differentiated cell, preferably at least 20%, more preferably at least 50% differentiated cell, of the desired cell type.

The term “Feeder cells” or “feeder layer” as used herein describes cells of one type that are co-cultured with cells of another type, to provide an environment in which the cells of the second type can grow. The compositions of the present invention are said to be “devoid of” or “free of” feeder layer if stem cells have been grown through at least one round after splitting without the addition of fresh feeder cells.

As used herein, the term “nutrient medium” refers to a medium for culturing cells, containing nutrients that promote proliferation. The nutrient medium may contain any of the following in an appropriate combination: isotonic saline, buffer, amino acids, serum or serum replacement, and other exogenously added agents and factors.

The term “genetically altered”, or “genetically transformed” cell is used herein when a polynucleotide has been transferred into the cell by any suitable means of artificial manipulation, or where the cell is a progeny of the originally altered cell that has inherited the polynucleotide. The polynucleotide will often comprise a transcribable sequence encoding a protein of interest, which enables the cell to express the protein at an elevated level. The genetic alteration is said to be “inheritable” if progeny of the altered cell have the same alteration.

The term “scaffold” as used herein refers to a supportive structural matrix which is more rigid than the HA-LN gel. The scaffold is not limited in its composition, shape, porosity, biodegradability and other physicochemical characteristics. The scaffold can give support to substances and compositions placed on its surface, embedded in its matrix, placed within its structure or placed at any other possible configuration.

As used herein, the term “biocompatible” refers to materials which may be incorporated into a human or animal body substantially without unacceptable responses of the human or animal. The term “biodegradable” refers to materials which, after a certain period of time, are broken down in a biological environment.

The present invention is based in part on the finding that cross-linked hyaluronic acid and laminin (HA-LN) gels can serve as a milieu for expanding stem cells in their undifferentiated state, replacing the dependence on a feeder layer, as to obtain a critical cell mass required for any therapy utilizing stem cells. Furthermore, the present invention discloses that under appropriate conditions, the expanded stem cells cultured in or on the HA-LN-Gel can differentiate. Differentiation of the stem cells can occur while the cells are cultured in or on the gel as well as when the expanded cell are transplanted into a body, particularly a mammalian body.

Stem cells used according to the teaching of the present invention can be pluripotent stem cells, capable of differentiating to any cell or tissue type, or partially committed stem cells, which are precursors of specific cell types.

HA-LN-Gel

The HA-LN-Gel has unique features including transparency, viscosity and adherence-support. The HA-LN-Gel provides a hydrophilic environment and facilitates sustained release of bioactive components. Advantageously, during the production of HA-LN-Gel compositions it is possible to control the viscosity and the degree of elasticity or malleability of the compositions, as well as other properties of clinical significance including but not limited to biodegradability, porosity and other attributes.

Specific compositions of HA-LN-Gels as well as methods for manufacturing this gel were described WO 02/39948 and U.S. application Ser. No. 10/669476 entitled “Cross-linked Hyaluronic Acid-Laminin Gels and Use Thereof in Cell Culture and Medical Implants” of some of the inventors of the present invention, the teachings of which are incorporated herein in their entirety.

According to one aspect, the present invention provides a composition for expanding stem cells, comprising a population of stem cells cultured in or on a biocompatible matrix comprising hyaluronic acid and laminin cross-linked to form a combined gel, wherein at least the majority of the cells maintain their undifferentiated state.

The present invention further discloses that the embryonic stem cells and expanded NOM can transform into a differentiation state in or on the HA-LN-Gel either in vitro under appropriate conditions or in vivo in a body, particularly a mammalian body.

General Techniques

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. For example, Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other texts are Creating a High Performance Culture (Aroselli, Hu. Res. Dev. Pr. 1996); Limits to Growth (D. H. Meadows et al., Universe Publ. 1974) and A Dissection and Tissue Culture Manual of the Nervous System (A. Shahar et al., Alan R. Liss. 1989). Tissue culture supplies and reagents are available from commercial vendors such as Gibco/BRL, Nalgene-Nunc International, Sigma Chemical Co., and ICN Biomedicals.

Animal treatment and maintenance are in accordance with the “Guide for the care and use of animals”, Institute of laboratory animal resources commission on life sciences, National Research Council, National Academy Press, Washington, D.C. 1966. DHEW publication no.80 (NIH) Office of Science and Health reports DRR/NIH, Bethesda, Md. 20205, USA. Cell biology, protein chemistry, and antibody techniques can be found in Current Protocols in Protein Science (J. E. Colligan et al. eds., Wiley & Sons); Current Protocols in Cell Biology (J. S. Bonifacino et al., Wiley & Sons) and Current Protocols in Immunology (J. E. Colligan et al. eds., Wiley & Sons.)

Sources of Stem Cells

The stem cells can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 1-2 weeks. For further details on methods of preparation human ES cells see for example U.S. Pat. No. 5,843,780, and Science 282: 1145, 1998. It will be appreciated that commercially available stem cells can be also be used with this aspect of the present invention. Human ES cells can be purchased from the NIH human embryonic stem cells registry (<http://escr.nih.gov>), UK Stem Cell (htt://www.nibsc.ac.uk); (http://www.mrc.ac.uk) and other commercially available resources. Non-limiting examples of commercially available embryonic stem cell lines are BG01, BG02, BG03, BG04, CY12, CY30, CY92, CY10, TE03 and TE32.

Stem cells used by the present invention can be also derived from human embryonic germ (EG) cells. Human EG cells are prepared from the primordial germ cells obtained from human fetuses of about 8-11 weeks of gestation using laboratory techniques known to a person skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until cell morphology consistent with EG cells is observed, typically after 7-30 days or 1-4 passages. For additional details on methods of preparation human EG cells see Shamblott et al., (Proc. Natl. Acad. Sci. USA 95: 13726, 1998) and U.S. Pat. No. 6,090,622.

Partially committed progenitor cells can be also used according to teaching of the present invention, including, but not limited to hematopoietic cell, neural progenitor cells, oligodendrocyte cells, skin cells, hepatic cells, muscle cells, bone cells, mesenchymal cells, pancreatic cells, chondrocytes and marrow stromal cells. According to certain currently preferred embodiments of the present invention, neural progenitor cells are obtained from nasal olfactory mucosa.

Olfactory mucosa comprises at least two anatomically distinct cell layers: olfactory epithelium (comprising of supporting cells, basal cells, immature neurons and mature sensory neurons) and lamina propria (comprising of ensheathing, glial cells, endothelial cells, fibroblasts and glandular cells). Olfactory ensheathing cells enwrap axons of olfactory nerves in olfactory nerve bundles in the lamina propria and in the olfactory bulb; the olfactory bulb is the site of olfactory nerve axon termination in the brain.

It will be appreciated that stem cells including partially committed stem cells for use according to the teaching of the present invention can be isolated from human tissues as well as from other species including mouse (Mills and Bradley, 2001, Trends Genet. 17(6): 331-9.), golden hamster (Doetschman et al., 1988, Dev Biol. 127: 224-7), rat (Iannaccone et al., 1994, Dev Biol. 163: 288-92) rabbit (Giles et al. 1993, Mol Reprod Dev. 36: 130-8; Graves & Moreadith, 1993, Mol Reprod Dev. 1993, 36: 424-33), several domestic animal species (Notarianni et al., 1991, J Reprod Fertil Suppl. 43: 255-60; Wheeler 1994, Reprod Fertil Dev. 6: 563-8; Mitalipova et al., 2001, Cloning. 3: 59-67) and non-human primate species including Rhesus monkey and marmoset (Thomson et al., 1995, Proc Natl Acad Sci USA. 92: 7844-8; Thomson et al., 1996, Biol Reprod. 55: 254-9).

Monitoring Cell Differentiation

The skilled practitioner will appreciate that there are a plethora of approaches well known in the art for monitoring pluripotency of cultured cells as well as the cell type of differentiating cells. For example morphological determination can be used to determine cellular differentiation. A number of morphological features are known to characterize undifferentiated stem cells such as high nuclear/cytoplasmic ratios, prominent nucleoli and compact colony formation with poorly discernable cell junctions.

Alternatively, cell differentiation can be determined upon examination of cell or tissue-specific markers which are known to be indicative of differentiation. For example, undifferentiated human embryonic stem cells are known to be immunoreactive with markers such as SSEA-3 and SSEA-4, GCTM-2 antigen, TRA 1-60, TRA 1-81 and telomerase reverse transcriptase (TERT). Similarly, neural progenitor cells may be characterized by expressed markers such as neuro ectodermal lineage; markers of neural progenitor cells; neuro-filament proteins; monoclonal antibodies including MAP2ab; glutamate; synaptophysin; glutamic acid decarboxylase; GABA, serotonin, tyrosine hydroxylase; β-tubulin; β-tubulin III; GABA Aα2 receptor, glial fibrillary acidic protein (GFAP), 2′,3′-cyclic nucleotide 3′-phosphodiesterase (CNPase), plp, DM-20, O4 and NG-2 immunostaining. Known markers for mature neural cells include but are not limited to MAP-2, neurofilament protein, glutamate, synaptophysin, glutamic acid decarboxylase (GAD), GABA, tyrosine hydroxylase and serotonin.

Examples of genes characteristic of pluripotent cells or particular lineages may include (but are not limited to) Oct-4 and Pax-6, polysialyated N-CAM, N-CAM, A2B5, nestin and vimentin as markers of stem cells and neuronal precursors respectively. Other genes characteristic of stem cells may include Genesis, GDF-3 and Cripto. CD-34 is characteristic of hematopoietic stem cells and flk-1 is expressed by the hemangioblast. AC-133 may be characteristic of both hematopoietic and neural progenitors. Keratin is characteristic of epidermal cells while transferrin, amylase and α1 anti-trypsin are characteristic of embryonic endoderm. Such gene expression profiles may be attained by any method including methods of differential gene expression, microarray analysis or related techniques.

The stem cells may be identified by being immunoreactive with markers for human pluripotent stem cells including SSEA-4, GCTM-2 antigen, TRA 1-60. Preferably the cells express the transcription factor Oct-4. The cells also maintain a diploid karyotype. Preferably the neural progenitor cells are identified by expressed markers of primitive neuroectoderm and neural stem cells such as N-CAM, polysialyated N-CAM, A2B5, intermediate filament proteins such as nestin and vimentin and the transcription factor Pax-6. Neurons may be identified by structural markers such as β-tubulin, β-tubulin III, the 68 kDa and the 200 kDa neurofilament proteins. Mature neurons may also be identified by the 160 kDa neurofilament proteins, Map-2a, b and synaptophysin, glutamate, GABA, serotonin, tyrosine hydroxylase, GABA biosynthesis and receptor subunits characteristic of GABA minergic neurons (GABA Aα2). Astrocytes may be identified by the expression of glial fibrillary acidic protein (GFAP), and oligodendrocyte by 2′, 3′-cyclic nucleotide 3′-phosphodiesterase (CNPase), plp, DM-20, myelin basic protein (MBP), NG-2 staining and O4.

Tissue/cell specific markers can be detected using immunological techniques known in the art (Thomson et al., 1998). Examples include but are not limited to flow cytometry for membrane-bound markers, immunohistochemistry for extracellular and intracellular markers and enzymatic immunoassay, for secreted molecular markers.

Differentiation of human ES cells in vitro is known to result in reduced expression of markers such as stage-specific embryonic antigens (SSEA) 3 and 4 and increased expression of others such as α-fetoprotein, NF-68 kDa, α-cardiac, Glut 2 and albumin.

In further embodiments, gene expression profiles may be used to determine cell phenotype. Relevant techniques well known in the art include but are not limited to RT-PCR, Northern Blot analysis and microarray analysis. For example, it is known that human embryonic stem cells express an elevated level of the transcription factor Oct-4. In contrast, neural progenitor cells do not express an elevated level of the transcription factor Oct-4 but rather are known to express an elevated level of the transcriptional factor Pax-6 as well as polysialylated N-CAM, N-CAM, A2B5, nestin, and vimentin.

Alternatively, immunofluorescence or immunocytochemical staining may be carried out on colonies of cells which are fixed by conventional fixation protocols then stained using antibodies against stem cell specific antibodies and visualized using secondary antibodies conjugated to fluorescent dyes or enzymes which can produce insoluble colored products.

Another approach to determine ES cell differentiation is effected via measurements of alkaline phosphatase activity. Undifferentiated human ES cells have alkaline phosphatase activity, which can be detected by fixing the cells with 4% paraformaldehyde and developing with the Vector Red substrate kit according to manufacturer's instructions (Vector Laboratories, Burlingame, Calif., USA).

The ability of ES cells to differentiate into cells of all three germinal levels (i.e., pluripotency) can also be used to monitor ES cell differentiation. Pluripotency of ES cells can be confirmed by injecting cells into SCID mice (Evans M J and Kaufman M 1983, Cancer Surv. 2: 185-208), which upon injection form teratomas. Teratomas are fixed using 4% paraformaldehyde and histologically examined for the three germ layers (i.e., endoderm, mesoderm and ectoderm). Alternatively, pluripotency of the stem cells of the present invention can be determined by their ability to form embryonal bodies.

In addition to monitoring a differentiation state, stem cells are often also monitored for karyotype, in order to verify cytological euploidity, wherein all chromosomes are present and not detectably altered during culturing. Cultured stem cells can be karyotyped using a standard Giemsa staining and compared to published karyotypes of the corresponding species.

It is also noted that differentiating cultures of the stem cells secrete human chorionic gonadotrophin (hCG) and α-fetoprotein (AFP) into culture medium, as determined by enzyme-linked immunosorbent assay carried out on culture supernatants. Hence this may also serve as a means of identifying the differentiated cells.

According to certain embodiments, the cells proliferate in the culture. In specific embodiments, at least some of the cells form a monolayer in the cell culture. According to other embodiments, at least some of the cells form an embryoid body structure in the cell culture. According to yet other certain embodiments, the cells keep their undifferentiated state through at least one passage of the cell culture, preferably through two to five passages.

Culture Medium

For the culture of stem cells, growth medium may be any growth medium suitable for growing the pluripotent or progenitor stem cells. The growth medium may be supplemented with nutritional factors, such as amino acids, (e.g., L-glutamine), anti-oxidants (e.g., beta-mercaptoethanol) and growth factors, which benefit stem cell growth in an undifferentiated state. After the cells reach a critical mass, the medium can be replaced to a growth medium including factors that promote differentiation into a specific cell type. When appropriate, serum and serum replacements are added at effective concentration ranges, as is known to a person skilled in the art. According to one embodiment, the culture medium is serum free. According to another embodiment, the culture medium comprises serum. According to certain currently preferred embodiments of the present invention, the serum is an autologous serum. According to another embodiment, the culture medium is enriched with at least one agent that supports the growth of the cells in an undifferentiated state. According to one embodiment, the agent is a growth factor selected from the group consisting of, but not limited to, basic fibroblast growth factor (bFGF), epidermal growth factor (EGF), members of the interleukin 6 (IL-6) and leukemia inhibitor factor (LIF).

According to yet another embodiment, the culture medium is enriched with at least one agent that induces differentiation and or promotes the growth of the differentiated cells. According to certain currently preferred embodiments, the culture medium is enriched with at least one agent that supports differentiation and growth of neuronal cells. According to one embodiment, the agent is selected from the group consisting of, but not limited to, brain-derived neurotrophic factors (BDNF), nerve growth factors (NGF), insulin-like growth factor-1 (IGF1), and leukemia inhibitory factor (LIF). Although LIF is known as a factor that keeps embryonic stem cell in an undifferentiated state, it can also serve as a factor that promote the long-term maturation of neuronal cell in culture.

The antioxidants N-acetyl-L-cysteine (NAC) and ascorbic acid (AA), and the protective compound pifithrin-α were found to be neuroprotective agents (both in vitro and in vivo). Slow release of these agents by the enriched HA-LN-Gel is therefore beneficial for the survival, growth and maturation of neurons in culture as well as after implantation.

Genetically Modified Stem Cells

According to one embodiment, the composition of the present invention comprises genetically modified stem cells. Typically, the cells are transformed with a suitable vector comprising an exogene for affecting the desired genetic alteration, as is known to a person skilled in the art.

The genetic alteration may be transient, or stable and inheritable as the cells divide. The genetically altered cells can be maintained in undifferentiated pluripotent form in culture, or they can be differentiated into other types of cells still retaining the genetic alteration.

The polynucleotide to be transferred in the cell typically provides a function that will change the phenotype of the cell or its progeny in a desirable fashion. For example, it may contain an encoding region under control of a promoter that promotes transcription in undifferentiated hES cells, or in differentiated cells of a particular lineage. It may also affect endogenous gene expression by a suitable mechanism, such as antisense reactivity, triplex formation, or ribozyme action.

Suitable methods for transferring vector plasmids into hES cells include lipid/DNA complexes, such as those described in U.S. Pat. Nos. 5,578,475; 5,627,175; and 5,705,308. Suitable viral vector systems for producing hES cells with stable genetic alterations are based on adenovirus and retrovirus, and may be prepared using commercially available virus components.

For many applications, genetic alteration of hES cells requires attention to two different agenda achieving sufficiently high efficiency of genetic alteration, and performing the alteration in a manner that does not promote differentiation of the hES cells along an undesired pathway. Screening of various transfection and transduction systems, and optimization of reaction timing and conditions, can be conveniently performed in experiments using an expression vector with an encoding region for a detectable label. Particularly convenient labels are intrinsically fluorescent, such as luciferase, or green fluorescent protein (GFP). The label may also be an enzyme that can be detected in histopathology or quantitated by enzyme reaction. Examples include alkaline phosphatase, and β-galactosidase. The label may also be a cell-surface protein that can be stained with labeled antibody and quantitated, for example, in a fluorescence activated cell counting device. Once an effective system has been identified and optimized, the encoding region for the label may then be substituted with the gene of interest.

Efficiencies of genetic alteration are rarely 100%, and it is usually desirable to enrich the population for cells that have been successfully altered. The genetically altered cells can be enriched by taking advantage of a functional feature of the new genotype. A particularly effective way of enriching genetically altered cells is positive selection using resistance to a drug such as neomycin or puromycin. To accomplish this, the cells can be genetically altered by contacting simultaneously with vector systems for the marker gene or gene of interest, and a vector system that provides the drug resistance gene. If the proportion of drug resistance gene in the mixture is low (3:1), then most drug resistant cells should also contain the gene of interest. Alternatively, the drug resistance gene can be built into the same vector as the gene of interest. After transfection has taken place, the cultures are treated with the corresponding drug, and untransfected cells are eliminated.

Uses of the Stem Cells

The stem cells cultured according to the teachings of the present invention can be used for several commercial and research applications.

Cultured stem cells obtained by the present invention can be used to prepare a cDNA library. The composition of the present invention allows the proliferation of the stem cells without the need for feeder layer, thus the cells are not contaminated with cDNA from feeder cells. mRNA is prepared by standard techniques from the pluripotent stem cells and is further reverse transcribed to form cDNA. The cDNA preparation can be subtracted with nucleotides from embryonic fibroblasts and other cells of undesired specificity, to produce a subtracted cDNA library by techniques known in the art.

Pluripotent or partially committed stem cells cultured according to the teachings of the present invention can be used to screen for factors (such as small molecule drugs, peptides, polynucleotides, and the like) or conditions (such as culture conditions or manipulation) that affect the characteristics of stem cells. For example, growth affecting substances, toxins or potential differentiation factors can be tested by their addition to the culture medium. As described herein above, the composition of the present invention has the advantage of being devoid of feeder layer, thus there is no interference caused by feeder cell.

In addition, there is a need for a system to enable transportation of stem cell cultures while not in a frozen state. The composition of the present invention, wherein the stem cells are embedded in a viscous environment, provides such system.

Furthermore, stem cells cultured according to the teaching of the present invention can be used for implantation, either per se, as a part of the composition of the present invention comprising hyaluronic acid and laminin cross-linked to form a combined gel, or within a scaffold as described herein below. According to certain embodiments of the present invention, neural progenitor cells isolated from adult nasal olfactory tissue or neural cells isolated from embryonic spinal cord cultured according to the teaching of the present invention are used for implantation.

Composite Implant

As disclosed herein above, the cells cultured in or on the HA-LN-Gel can be used for clinical therapy per se, or as a part of the composition comprising the gel. Additionally, the compositions of the present invention can be further used for the formation of a composite implant, which further comprises a scaffold.

It was disclosed previously by some of the inventors of the present invention (WO 02/39948) that the HA-LN-Gel serves as a highly advantageous biocompatible implant and as delivery vehicle for transplantation. Nevertheless, it was further disclosed by some of the inventors of the present invention (WO 2004/029095, entitled “Cohesive Coprecipitates Of Sulfated Polysaccharide And Fibrillar Protein And Use Thereof”, the teachings of which are incorporated herein their entirety) that in order to improve its mechanical properties it is desirable to support the HA-LN-Gels with a more rigid scaffold prior to implantation into a patient.

Any appropriate scaffold for supporting the cells expanded on a hyaluronic acid-laminin gel may be employed. WO 2004/029095 discloses compositions comprising coprecipitates of at least one sulfated polysaccharide and at least one fibrillar protein, exemplified by a coprecipitate of dextran sulfate and gelatin, that form a cohesive biopolymer having unique physicochemical attributes useful as universal biomatrices or scaffolds for clinical applications, including as implants for tissue engineering.

The cohesive biopolymer is prepared by combining the sulfated polysaccharide and the fibrilar protein in a solution having an acidic or basic pH, in the presence of a volatile organic solvent. These conditions can cause denaturing of the fibrilar protein, as to result in a coprecipitate of the fibrilar protein and sulfated polysaccharide that has unique physicochemical properties If a strong matrix is desired, cross-linking agent can be further added. When dextran sulfate and gelatin are used, the scaffold properties would be essentially determined by the type of the dextran sulfate used (high or low molecular weight) and the pH of the reaction.

The scaffold can be shaped to various forms as to support the cell-bearing HA-LN-Gel. As exemplified herein below, the scaffold can be shaped in a form of a tube, which further comprises nanofibers made of the coprecipitate, wherein the tube encloses the cell bearing HA-LN.

Both the HA-LN-Gel and the dextran sulfate-gelatin scaffold are biocompatible, i.e. do not evoke significant adverse effects when incorporated into a human or animal body, and are therefore highly suitable for use in implantation. Furthermore, the ability to enclose the cells for transplantation within these structures can significantly reduce the induction of an immunogenic response against the transplanted cells by the receiving body.

According to yet another aspect, the present invention provides a method for transplanting cells to an individual in need thereof, comprising the step of transplanting a composite implant comprising cells cultured in or on cross-linked hyaluronic acid-laminin gel, further comprising a biocompatible scaffold wherein the scaffold supports the cell culture. According to certain embodiments, the cells are transplanted into a site of an injured tissue. According to certain currently preferred embodiments, the cells are transplanted into an injured site of spinal cord tissue. According to one embodiment, the method further comprises covering the site of an injured tissue with a thin biodegradable membrane for fixation of the implants at the injured site. According to one embodiment, the membrane comprises a coprecipitate of dextran sulfate and gelatin. According to another embodiment, the membrane is attached to the injured site by interstitial sutures. This membrane can be made grooved and positively charged to enable cell attachment and guidance of the regenerated axons.

The cells within the implant can be undifferentiated stem cells, differentiated cells or a combination thereof. Furthermore, the implant can comprise cells of the same type as well as a plurality of cell types.

According to certain currently preferred embodiments, the composite implant used according to the teaching of the present invention for transplanting cells into an injured site of a spinal cord comprises cells selected from the group consisting of partially committed cultured adult progenitor NOM cells, embryonic spinal cord neural cells or combinations thereof. The cells can be of autologous or allogeneic source.

Regeneration and repair of partial and complete incision injuries of the spinal cord is still an unresolved clinical challenge. Various experimental approaches for reconstructive regeneration and renewal of damaged spinal cord are known in the art, including stimulation of positive autoimmune responses, introduction of neurotrophic and neuroprotective agents, or removal and elimination of scar inhibitory molecules. The latest technologies for solving paraplegic conditions employ several kinds of cell therapy that are introduced into the damaged site of the spinal cord after removal of the accumulated scar, for example implantation of tissue-engineered devices, without cells, for anchorage of the implant and for guiding axonal regeneration and use of composite implants, containing cells of either autologous or allogeneic origin. The choice of cell resources involves implantation of either stem cells directed to differentiate toward mature neurogenic phenotypes, or insertion of already mature neuronal committed cells.

As exemplified herein below, the compositions and methods of the present invention can be used with both cell sources. The compositions and methods for expanding stem and progenitor cells of the present invention serve the purpose of establishing high concentrations of progenitor as well as differentiated neural cells. These cells are embedded in a milieu of a HA-LN-Gel enriched with adhesive molecules and neurotrophic and neuroprotective agents as antioxidants, which are released slowly. The cell bearing gel is further supported by a scaffold. Thus, the compositions and methods of the present invention answers the limitations of hitherto known implantation methods by providing a system for supporting cell expansion and differentiation as to provide sufficient amounts of the desired cells, together with the means for anchoring the cells into a specific site of an injured tissue, specifically into a site of transected spinal cord.

In addition, a variety of cells sources for implantation are known in the art, including bone marrow stroma cells, skin, umbilical cord blood and embryonal and fetal stem cell lines. However, those cell sources have proven to support only sporadic appearances of single cells, or cell aggregates of neuronal cells, and failed to yield robust numbers of neuronal cells to create a successful implant that can replace massive segmental losses. As exemplified herein below, embryonic spinal cord and adult NOM cells showed vigorous vitality that established rich neurogenic cell cultures for implantation.

### EXAMPLES

Materials and Methods

(i) H-LN-Gel

Hyaluronic acid-laminin gel (HA-LN-Gel) is described in details in WO 02/39948, incorporated herein in its entirety by reference. The gel is composed of cross-linked hyaluronic acid with the adhesive molecule laminin, and the following mixture of ingredients: antioxidants, neuronal growth factor; neuro-protective factors such as EGF, bFGF, BDNF and NGF (20-50 ng/ml), IGF-I (50 ng/ml), LIF (0.5 u/ml), NAC (n-acetyl cystein, 10 μM), pifithrin α, cyclic (200 nM) and retinoic acid (1-5 μM).

HA-LN-Gel is transparent, highly hydrated with polar and non-polar (hydrophobic) sugar residues, all biocompatible and biodegradable. For cell cultivation, the gel is used at a concentration of 0.7-1% hyaluronic acid. For implantation a more viscous gel (1.2-1.5%) is used.

(ii) Stem Cells

Undifferentiated human embryonic stem cells (NIH approved) were grown on mouse embryo fibroblasts as previously described (Amit et al., 2000. Dev. Biol. 227:271-278), i.e. in serum free culture medium or in medium with 20% fetal calf serum (FCS). The primary cultures were dissociated with collagenase IV and cells were embedded in HA-LN-Gel, supplemented with 5 ng/ml leukemia inhibitor factor (LIF) and 4 ng/ml basic fibroblast growth factor (bFGF), in undifferentiating serum free medium.

Bovine blastocytes grown for about a month either as clusters of embryonic cells in nutrient medium covered by a drop of oil, or as a dissociated cell culture were provided by IMT Company (Ness Ziona, Israel). The surrounding zona pelicuda was manually removed, and the inner cell mass (ICM) was enzymatically dissociated and seeded in HA-LN-Gel (containing serum replacement medium or medium supplemented with serum and factors). After few days as stationary cultures in the gel, the cells were further dissociated and re-seeded in the gel.

(iii) Expansion Media

Expansion media consisted of 80% KnockOut® DMEM (an optimized Dubecco's modified Eagle's medium for ES cells; Gibco-Invitrogen, San Diego, Calif.), 20% KnockOut® SR (a serum replacement formulation; Gibco), 0.2% antibiotic-antimycotic (Gibco), 1 mM glutamine (Gibco), 0.1 mM β-mercapto ethanol (Sigma, St. Louis, Mo.), 1% non-essential amino acids (NEA) (Gibco), 5 ng/ml leukemia inhibitor factor (LIF) (Sigma) and 4 ng/ml basic fibroblast growth factor (bFGF) (PeproTech Inc. Rocky Hill, N.J.).

(iv) Enzymatic Dissociation Mixture

RDB, a mild proteolytic vegetal enzyme, a courtesy of Dr. David Ben-Nathan (Dev Biol Stand. 1985; 60:467-473), was initially diluted 1:30 and was further diluted 1:1 with 0.05% EDTA (Biological Industries, Beit-HaEmek, Israel). Alternatively, the enzymatic dissociation mixture contained at least one of collagenase, DNAae Trypsin, Trypsin-like vegetative enzyme and combinations thereof.

(v) Scanning Electron Microscopy (SEM)

Cultures were fixed in 2.5% glutaraldehyde for 2 h and washed three times with distilled water. Dehydration was accomplished in increasing concentrations of ethanol. Critical point drying was performed using liquid CO₂ and a Polaron drying apparatus. Specimen was vacuum coated with 100-200 Å layer of gold (Polaron spattering unit) and was observed using Jeol 5410 LV SEM at 25 kV.

(vi) Fluorescent Staining

Cultures or cover slips were rinsed once with PBS and then fixed with 4% paraformaldehyde (in phosphate buffer pH 6.0) for 10 minutes. Permeabilization was performed for 10 minutes at room temperature using 0.5 % Triton-X-100 (in phosphate buffer pH 6.0). Cells were further fixed for additional 10 minutes and washed three times in PBS before incubation with antibodies. Antibodies against Neural cell markers were used: microtubule associated protein (MAP-2) (rabbit, polyclonal; Chemicon, Temecula Calif., diluted 1:50) and neurofilament (NF) protein (mouse, monoclonal; Dako, Danmark, diluted 1:50). Secondary antibodies were Cy₂™ or Texas Red conjugated goat anti mouse or rabbit IgG, (Jackson, West-Grove, Pa., diluted 1:100). Fluorescent samples were observed using Olimpus microscopes (IX70 inverted or AX upright) and photographed using an Optronix Magnafire camera.

(vii) Cultivation of Adult NOM and Embryonic Spinal Cord Cells

Biopsies of adult human olfactory nasal mucosa and embryonic spinal cords of aborted fetuses (16-23 weeks of gestation) were collected for cell isolation and the establishment of cultures. The study with human derived samples was approved by the Helsinki committee of Assaf-Harofeh Medical Center (#13/03). Informed consent was obtained from each patient.

The culturing method combines stationary cultures in the HA-LN-Gel, alternating with cells grown in suspension on an anion exchange, positively charged cylindrical, DEAE-cellulose (DE-53) microcarriers (MCs), (Whatman, England). The MCs are equilibrated with phosphate buffered saline (PBS) pH 7.4, and autoclaved in batches of 15 g in 100 ml PBS essentially as previously described (Shahar A. 1990. Methods in neurosciences 2: 195-209; Goldman S A, et al., 1997. Ann N Y Acad Sci 835:30-55).

The dissociated cells were grown in suspension attached to the MCs for periods of 1-4 weeks. At various times during their growth period in suspension, cell-MC aggregates were collected and reseeded in the HA-LN-Gel as stationary cultures. The following growth media were used: modified Neuronal Epithelial Progenitor (NEP) medium or M-21 medium. NEP medium contains DMEM-F12 (Invitrogen), N2 additives (progesterone, putresine, selenium, insulin, transferrin), B27 supplements (Invitrogen) and 1% BSA. M-21 medium is based on NEP medium, except that B27 is omitted and 100 μM nonessential amino acids, 30 ng/ml triiodothyronine and 1 mM sodium pyruvate are added. Both media were supplemented with the same factors used to enrich the gel. For cell expansion, the media were enriched with 10% fetal calf serum (FCS); for growth differentiation and implantation the serum was omitted from the media. Cells were usually cultured for 3-4 weeks prior to implantation.

(viii) Scaffold

Cohesive co-precipitate of dextran sulfate and gelatin is described in WO 2004/029095 incorporated herein in its entirety by reference. The co-precipitate used as scaffold (designated NVR-scaffold) has a tubular format with a customized diameter of 2 mm and a wall thickness of 0.4 mm. In addition, the tubular scaffold contains a bundle of parallel nanofibers 50-100 μm in diameter, made of the same material as the scaffold (FIG. 5). The scaffold is biocompatible, non-toxic and non-inflammatory. The tube is transparent, suturable and it can last for a period of more than three months until biodegradation takes place.

(ix) Surgical and Transplantation Procedure

The study was authorized by the local ethical committee for experiments in laboratory animals. Animal treatment and maintenance were in accordance with the “Guide for the care and use of animals”, Institute of laboratory animal resources commission on life sciences, National Research Council, National Academy Press, Washington, D.C. 1966. DHEW publication no. 80 (NIH) Office of Science and Health reports DRR/NIH, Bethesda, Md. 20205, USA.

Twenty Sprague-Dawly rats, three-months-old, each weighing approximately 250 gr, were used. All rats were anesthetized by intraperitoneal injections. For the operations, anesthesia consisted of Ketamine HCL (50501 USA) 125-130 mg/kg and Xylosine (B2370 Belgium) 4.8 mg/kg. For electrophysiological tests, rats were anesthetized for a short period (30 min.) with Ketamine 75-85 mg/kg and Xylosine 3 mg/kg. For removal of stitches, light anesthesia was used: 50 mg/kg Ketamine and 1.8 mg/kg Xylosine. All the treatments and the follow up tests were performed in a double-blind randomized manner.

All surgical procedures were performed on anesthetized rats, using a high magnification navigator microscope (Zeiss NC-4) in a class 100 animal operating room. The spinal cord was exposed via a dorsal approach. The overlying muscles were retracted, T7-T8 laminae were removed, the spinal cord was completely transected using micro-scissors and a 4 mm segment of the cord was removed. The 4 mm gap was chosen to match the gap size formed after removal of the scar in chronic spinal cord injuries.

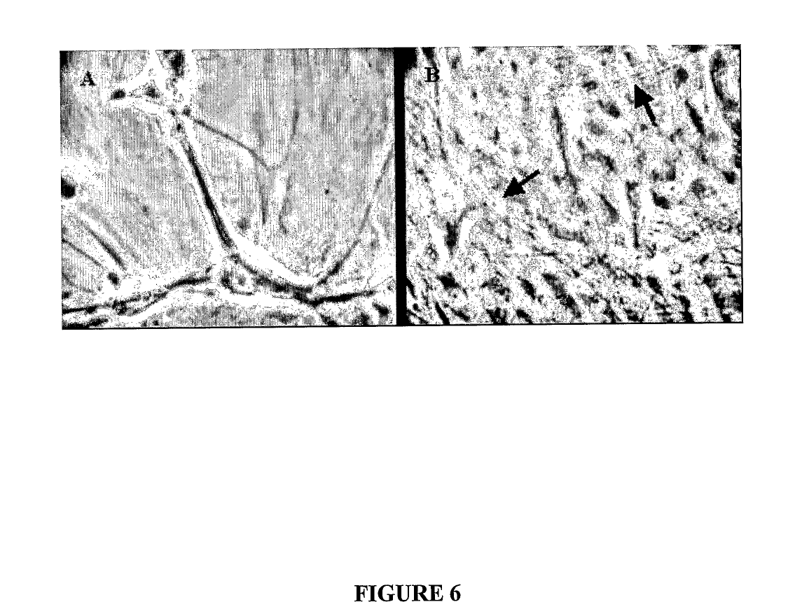

The composite implant was prepared as follows: embryonic human spinal cord cells were grown as long term cultures to the stage of myelin formation (FIG. 6). About 1-2×10⁶ NOM or spinal cord cells, embedded in HA-LN-Gel, and encapsulated in NVR scaffold, were implanted into the site of the excised spinal cord segment.

(x) Post-Operative Animal Maintenance

In the post-operative management of the animals care was taken to minimize discomfort and pain. Following implantation the rats were assisted in urination and defecation with the help of a veterinarian, twice daily. Animals were maintained in ventilated cages, containing sawdust and sterile food. The paraplegic rats were kept solitary in cages, but were gathered in groups for one hour every day, in a large facility. At the termination of the experiments, the animals were sacrificed under general anesthesia.

(xi) Electrophysiological Measurements

Somatosensory evoked potentials (SSEP) were recorded in the experimental and control groups in a blinded manner, immediately postoperatively and 3 months later. Conductivity of the spinal cord was studied by stimulation of the sciatic nerve and recording from two disc-recording electrodes, active and reference, placed on the rats' scalps. These electrodes, with conductive jelly, were attached to the scalp-active over the somatosensory cortex in the midline and reference electrode between the two eyes. The earth electrode was placed on the thigh, on the side of the stimulation. The sciatic nerve was stimulated by a bipolar stimulating electrode. Two hundred and fifty-six stimulation pulses of 0.1 msec in duration were generated at a rate of 3/sec. The stimulus intensity was increased gradually, until slight twitching of the limb appeared. The appearance of evoked potentials as a response to stimulation in two consecutive tests, was considered positive. Latency and amplitude (positive—P wave peak) were measured.

The rats were anesthetized intra-operatively with diluted Nembutal 15 mg/kg weight. The test was performed using the Medelec/Teca Sapphire 4 ME electromyography apparatus (20 Hz to 2 KHz band pass filter and calibration sensitivity 10-20 mcV/div and time base 5 ms/div).

(xii) Locomotor Rating Scale

The Basso, Beattie, Bresnahan (BBB) open field locomotor scale is a popular measure of functional recovery following spinal cord injury (Basso D M, et al., 1995. J Neurotrauma 12: 1-21). The BBB is graded from 0 (absent performance) to 21 (complete-normal gait performance). This grading scale was used to assess behavioral recovery and gait performance weekly for 2-10 months after spinal cord injury.

(xiii) Sample Preparation for Magnetic Resonance Imaging (MRI)

Spinal cords were excised and fixed with formalin. The spinal cords were inserted into 5 mm NMR tubes with their long axis parallel to the z-direction (the Bₒ direction) of the magnet and immersed in Fluorinert (Sigma Chemical Co., St. Louis, Mo.). The temperature in the magnet was maintained at 25.0±0.1° C. for the duration of the experiments.

(xiv) MRI Experiments

MRI diffusion experiments were performed on a wide-bore 8.4 T NMR spectrometer (Bruker, Karlsruhe, Germany) equipped with a micro5 imaging gradient probe (Bruker, Karlsruhe, Germany) capable of producing pulse gradients of up to 190 gauss cm⁻¹ in each of the three directions. Diffusion weighted MR images were collected using the stimulated-echo diffusion imaging pulse sequence with the following parameters: TR=2000 ms, TE=35 ms, δ=3 ms and Δ=50 ms. The diffusion gradient strength, G, was incremented from 0 to 60 gauss/cm in 16 steps giving a maximal b value of 1.12×10⁶ s cm⁻² and qₘₐₓ of 766 cm⁻¹. Diffusion was measured perpendicular and parallel to the long axis of the spine. MR images were collected in a blind mode and the trauma site was placed at the center of the imaged region.

The signal decay of water was analyzed using the q-space approach (Cory D G, and Garroway A N. 1990. Magn Reson Med 14: 435-44) using the Matlab program.

(xv) Histological Analyses

Prior to cultivation, a small segment of the NOM biopsy was taken for histological staining after fixation in 10% formalin at pH 7.4.

The spinal cord area, containing the implant region with both the proximal and the distal normal healthy stumps, was fixed as a whole mount in 10% formalin for several days. Subsequently, samples were placed in ethylenediaminetetra-acetic acid (12.5%) at pH 7.0 for decalcification. The softening of the calcified bony spine enabled the smooth release of the whole region of the spinal cord, which could be inserted into the tube probe of the MRI system (described later). For histological analysis the spinal cord was dissected into 7 sections of 3-4 mm each, in parallel with the MRI analysis. The pieces were rinsed thoroughly in running tap water. The samples were dehydrated in sequential alcohols, xylol and embedded in paraffin. Sections of 5-6 microns were re-hydrated and stained with Meyer's hematoxylin-eosin, Masson's trichrome and Bodian silver methods (Pearse A. 1972. Histochemistry Theoretical and Applied. 3rd ed. Vols 1&2. Boston, Mass: Little Brown and Co).

(xvi) Immunofluorescence

Cultures were fixed with 4% paraformaldehyde and incubated with antibodies against epitopes of Olfactory Marker Protein (OMP was kindly provided by Prof. F. Margolis, Univ. of Maryland, Baltimore, Md., USA) and microtubulin associated protein-2 (MAP-2). Cells were then washed and incubated with Cy™2 or Texas Red conjugated goat anti-mouse or rabbit IgG (Jackson, West-Grove, Pa., diluted 1:100). The stained cultures were inspected under fluorescent microscope (Olympus, Japan) with suitable filters and photographed using Magnafire SP digital camera (Optronix, USA). For positive control, rat neuronal cultures were stained under the same conditions.

(xvii) Study Initiation and Nasal Biopsy from Chronic Paraplegic Dog

Eligible dogs that need surgery at presentation (time 0) are operated as soon as possible (e.g. reduction and fixation of vertebral bodies). From time 0 and every other day throughout the whole experiment period, the dogs are undergo a neurological evaluation, the results of which are documented by means of digital recording. They are submitted to the following exams which are taken during the 2 months prior to implantation: haematology, urine status and liquor (cervical collection) magnetic resonance imaging (MRI), weighted, somato-sensory evoked potentials (SSEP), motor evoked potentials (MEP), H reflex, and urodynamic functions.

During the first month after presentation, a NOM-cell biopsy is taken by rhinoscopy from the central deep turbinate of the left nose. The procedure is performed under premedication with Domitor and Propofol, and general anaesthesia with Isofluorane. The NOM cells biopsies inserted in a special medium is delivered to Neural & Vascular Reconstruction laboratory within 72 hours for further processing and cultivation as described bellow. After four weeks, the composite implant is delivered to the surgery facilities in Sordio, Camerino and Bern.

(xviii) Surgical Procedure in Chronic Paraplegic Dog

A routine approach to the injured spinal cord by dorsal laminectomy is preformed. The surgeon removes the injured segment of the spinal cord including bone scar and/or fragments, intramedullary cavities, glial connective tissue (scar tissue) and periferic connections with healthy spinal cord. The spinal cord is sectioned at this part of the surgical area through the lower limit of the dura mater. An indication to the healthy parts may be given by consisting bleeding from the Ventral Spinal Artery (unpaired vessel in the median fissure), the Vertebral Venous Sinuses (paired, on the floor of the vertebral canal in the epidural fat) and from the spinal veins (paired vessels which follow the nerve roots to the intervertebral space and into the vertebral venous sinuses). All the scar tissues thoroughly removed are preserved for histological analysis. Surgery is done in a microsurgical manner using a magnification device and micro instrument in order to limit the destruction of normal tissue.

An implantation of the composite implant is performed into the residual gap after homeostasis has been achieved. An additional 0.2-0.5 ml of the composite implant is infiltrated under the dura (bridging method) using a 22 gauge flexible catheter. The dura is closed using a dural graft matrix (DuraGen). A low power laser irradiation He—Ne 632.8 mn is used directly on the implant for 10 minutes at a distance of 2 cm in order to promote neuronal regeneration by the activation of inflammatory cells (clear myelin debris and secrete regeneration factors), reducing cavitations and contrasting edema. Finally closure of muscles and skin is preformed.

After the implantations the dogs are undergoing frequent neurological evaluations, the results of which are documented by means of digital recording. They are submitted to the following exams: haematology and urine status—once every other week, liquor—after 1, 3 and 6 months; evoked potentials every 45 days and aerodynamic study after 1, 3 and 6 months. Spinal MRI-1 week, 1 month, 3 months, and 6 months after implantation.

Physical treatments and hydrotherapy are followed the protocol and include physiotherapy: massages, passive movement, PNF (proprioceptive neuromuscular facilitation), reflex induced training (stimulation of withdrawal reflex and extensor reflex) and active movement (standing, isometric exercises and gait exercises). Additionally the management of the bladder and prophylaxis of decubital ulcers and dermatitis are part of the rehabilitation program.

### Example 1

### Pluripotent Human Embryonic Stem Cells (hES) Cultures in a HA-LN-Gel

Undifferentiated hES cells (H9.2 clone) were grown on mouse embryo fibroblasts as previously described (Amit et al, supra). Briefly, the cells were grown in a serum free culture medium or in medium with 20% fetal calf serum (FCS). The primary cultures were dissociated with collagenase IV.

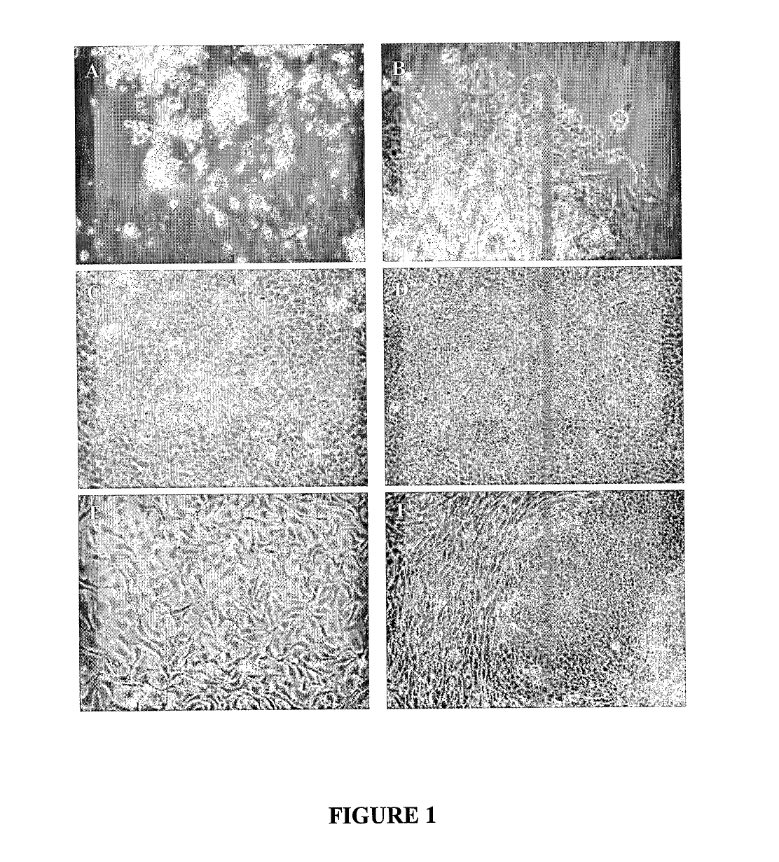

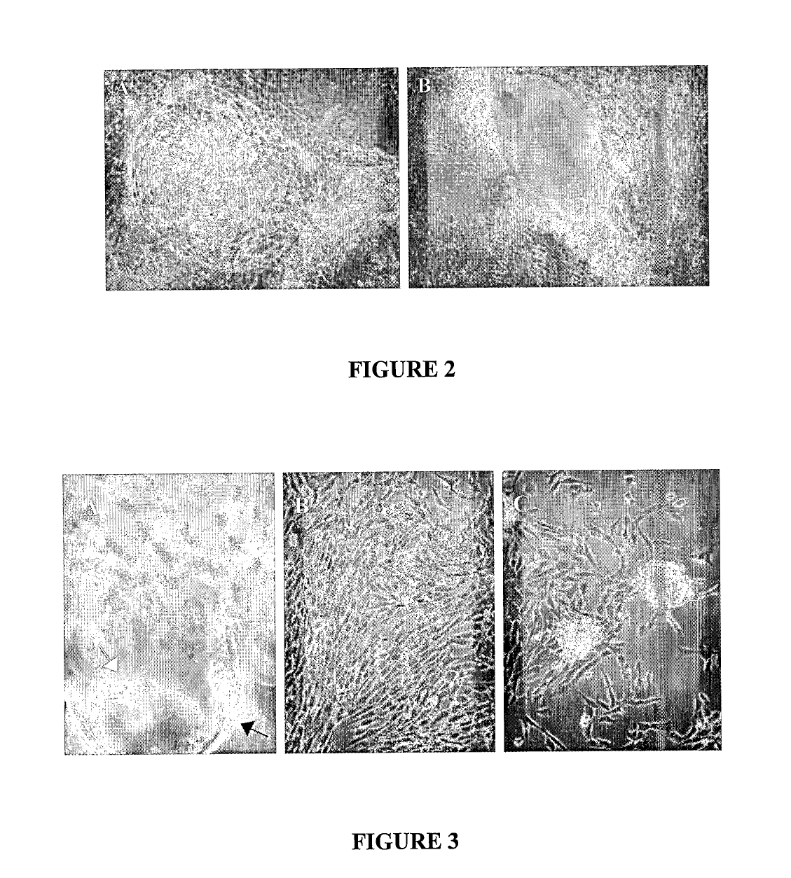

Subsequently, cells were embedded in the HA-LN-Gel, and cultured without a feeder layer in the Expansion Media described above. In the gel milieu, cells exhibited intensive growth of both epithelial-like cells, which actively divided forming a monolayer (FIG. 1) and embryoid bodies (EB)-like constructs that grew in a three-dimensional pattern (FIG. 2). These cultures were grown in HA-LN-Gel for 10 weeks; during this time several cultures were further dissociated and reseeded in HA-LN-Gel. Differentiated cells could not be detected at this stage.

FIG. 1 shows micrographs of human ES cells grown in HA-LN-Gel. Cells expanded in the gel and formed monolayers of epithelial and endothelial like cells of different shape. Section A displays hES cell-aggregates soon after embedding in HA-LN-Gel. Section B shows a micrograph of the hES cells 15 hours after embedding in the HA-LN-Gel. Sections C-E show micrographs of the hES cells 8, 22 and 24 days after embedding in the HA-LN-Gel. Section F shows a micrograph of the hES cells 24 days after embedding in the HA-LN-Gel. In Sections C-F, formed monolayers are visible, showing cells which differ in size and shape. Original magnification: sections A, D-F-200×, sections B&C-400×.

FIG. 2 shows micrographs of hES cells grown for 22 days in serum free medium in the HA-LN-Gel. Clusters of hES cells exhibiting three-dimensional growth are visible. Original magnification: section A-200×, section B-100×.

### Example 2

### Cultivation of Multipotent Precursor Cells from Bovine Blastocyte

Bovine blastocytes grown for about a month either as clusters of embryonic cells in nutrient medium covered by a drop of oil, or as a dissociated cell culture were provided by IMT Company. The surrounding zona pelicuda was manually removed, and the inner cell mass (ICM) was enzymatically dissociated and seeded in HA-LN-Gel (containing serum replacement medium or medium supplemented with serum and factors). After few days as stationary culture in the gel, the cells were further dissociated and re-seeded in HA-LN-Gel.

FIG. 3 shows micrographs of bovine blastocytes grown in HA-LN-Gel. Inner cell mass (ICM) of bovine blastocyte (white arrow) inside the remains of the zona pelicuda (black arrow) 1 day after seeding in HA-LN-Gel; original magnification: 400× (section A). Cellular growth and migration from the ICM, two weeks after seeding in HA-LN-Gel; original magnification: 200× (section B). Growth of undifferentiated cells 4 days after enzymatic dissociation and re-seeding in HA-LN; Original magnification: 200× (section C). The inventors managed to grow cells from bovine blastocytes in HA-LN-Gel for several weeks.

### Example 3

### Cultivation of Umbilical Cord Blood Stem Cells

Umbilical cord blood was collected immediately following delivery into 50 ml polystyrene tubes containing heparin. Fresh blood (up to 4 hours on ice) was subjected to FICOL gradient to remove red blood cells (RBC). At this stage, cord blood stem cells were embedded in HA-LN-Gel, in RPMI medium supplemented with 10% bovine serum.

Alternatively, after FICOL gradient, different populations of WBC were separated based on their expression of surface molecules CD34 and CD133, using magnetic beads and the kit of Milentni BioTech. Different populations of cells (i.e. CD34+, CD34−, CD133+, CD133−) were seeded in HA-LN-Gel as described above.

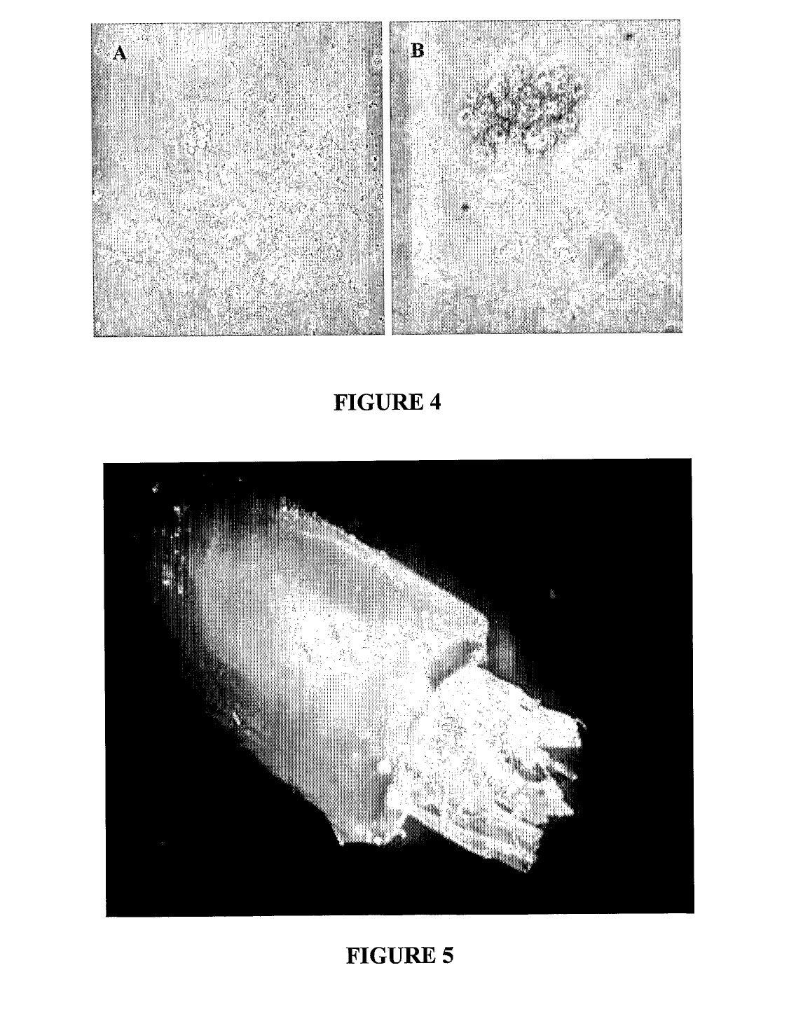

HA-LN-Gel was found to support the growth and replication without differentiation of the various populations of WBC isolated from umbilical cord blood (FIG. 4, sections A and B). Aggregates of round and dividing cells were observed for a period of several weeks, without the presence of feeder layer or conditioned medium.

### Example 4

### Cultivation of Adult Progenitor NOM and Embryonic Spinal Cord Cells

Biopsies of adult human olfactory nasal mucosa and embryonic spinal cords of aborted fetuses (16-23 weeks of gestation) were collected for cell isolation and the establishment of cultures. The biopsies were enzymatically dissociated and the remaining tissues were manually removed. The cells were seeded in HA-LN-Gel (containing serum replacement medium or medium supplemented with serum and factors). After few days as stationary cultures in the gel, the cells were further dissociated and grown in suspension on an anion exchange, positively charged cylindrical, DEAE-cellulose (DE-53) microcarriers (MCs), and then re-seeded in the HA-LN-Gel, as described herein above.

Embryonic human spinal cord cells were grown as long term cultures to the stage of myelin formation (FIG. 6).

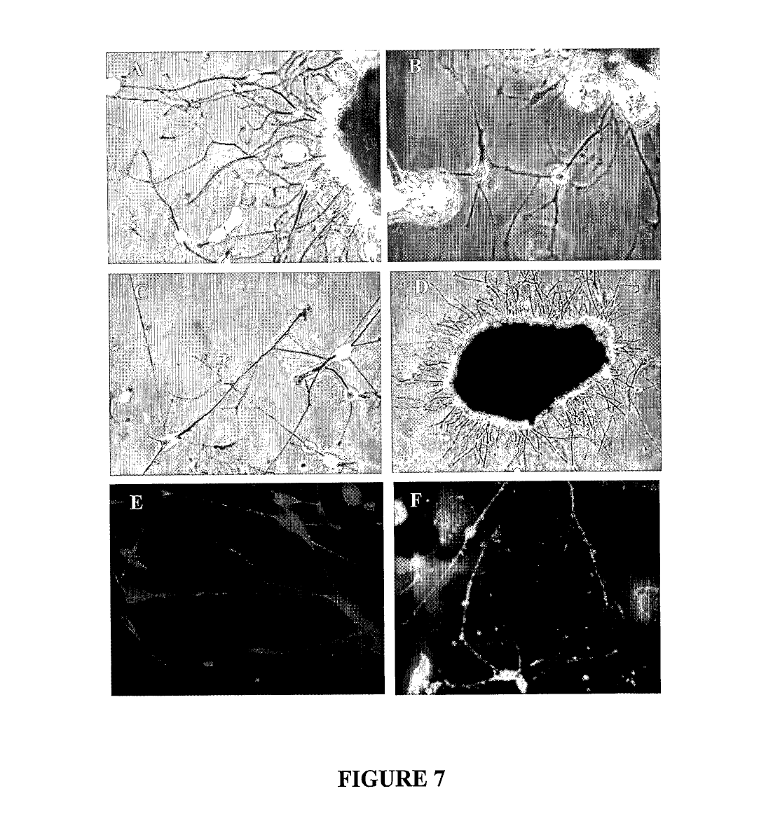

Adult human NOM cells were successfully grown and expanded. A large number of neuronal cells were observed among epithelial cells organized in large laminae and tapered cells forming islands of confluent cells, in the cultures that were reseeded in HA-LN-Gel, following a growth period of 1-2 weeks in suspension on positively charged microcarriers (MCs). (FIG. 7).

### Example 5

### Implantation of a Composite Comprising NOM or Embryonic Spinal Cord Cells for the Treatment of Traumatic Spinal Cord Injury in Rats and Post Operative Observations

All surgical procedures and spinal cord transections were performed on twenty Sprague-Dawly rats as described in section (ix) of Materials and Methods.

Eight of the twenty rats that underwent spinal cord transection and the removal of a 4 mm spinal cord segment were closed with no further treatment (sham treated control group). The remaining 12 rats underwent implantation of one of the composite implants. Eight rats were treated with implants containing NOM cells and 4 rats received human embryonic spinal cord-derived cells.

Four mm long composite implants were placed in the transected area of the spinal cord, in direct contact with the margins of the two stumps. The entire area of the lesion containing the implant was covered with a thin biodegradable membrane composed of the dextran-sulfate coprecipitate, attached by a few interstitial sutures for fixation of the implants at the desired sites. Finally, the muscular and cutaneous planes were closed and sutured.

Seven animals out of 8 in the sham treated control group exhibited complete paraplegic characteristics on physical examination and in their gait performance analysis (BBB=0: No observable hind limb movement). One rat of the control group showed slight movement of the hip joints (BBB=1).

Three out of the 8 rats in the group treated by a composite implant containing cultured human nasal olfactory mucosa (NOM), showed varying degrees of active movements. One of the three rats showed BBB=3 (extensive movement of hip and knee joints), one rat showed BBB=10 (occasional weight-supported plantar steps; no FL-HL coordination) and one rat showed BBB=13 (frequent consistent weight-supported plantar steps and FL-HL coordination) (Table 1).

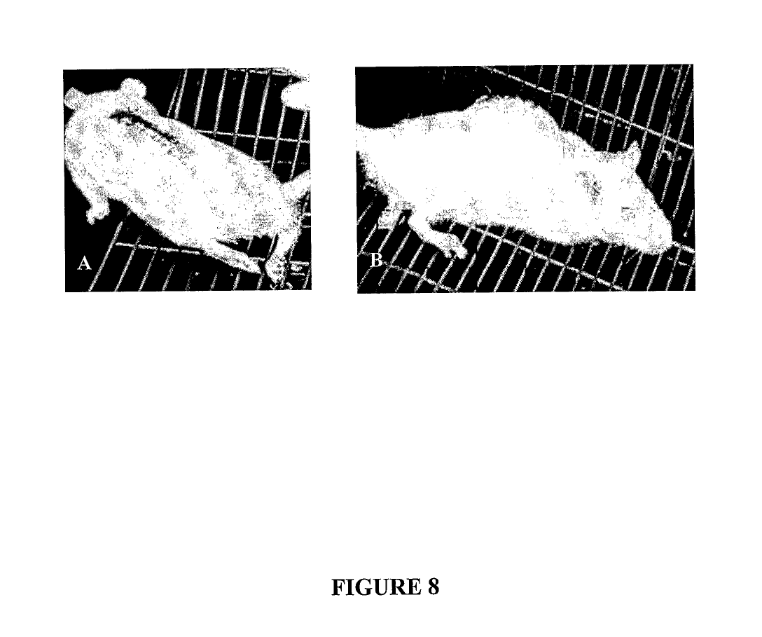

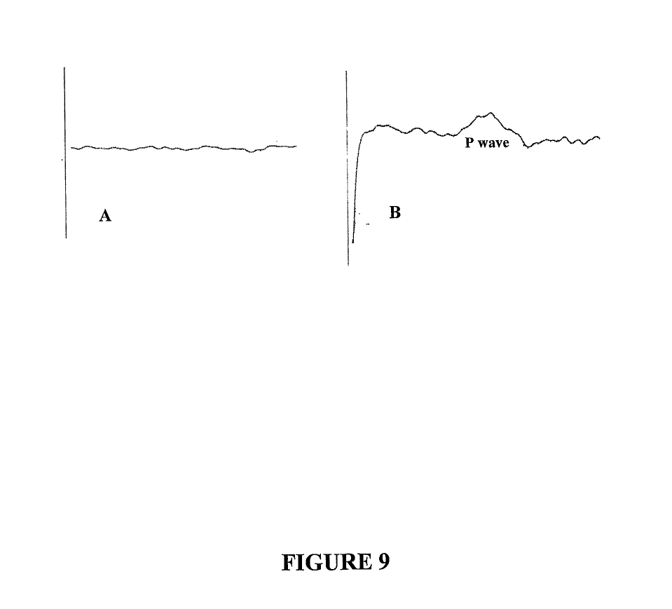

All 4 rats in the group treated by human embryonic spinal cord cells implantation showed varying degrees of leg movements: one rat showed BBB=3 (extensive movement of hip and knee joints), one rat showed BBB=6 (extensive movement of two joints and slight movement of the third), one rat showed BBB=10 (occasional weight-supported plantar steps; no FL-HL coordination) and one rat showed BBB=13 (frequent consistent weight-supported plantar steps and FL-HL coordination). FIG. 8 shows a control rat that underwent complete transection of the spinal cord and removal of a 4 mm segment, that is completely paralyzed in both legs, folded inward (FIG. 8, section A), and paraplegic rat showing restoration of partial gait performance (in the right leg) three weeks after implantation of a composite implant containing cultured adult human NOM cells into a 4 mm gap of transected spinal cord (FIG. 8, section B).

TABLE 1
BBB locomotor rating scale of the three groups of animals, two
experimental and one control

| | | | Human | | | | | |
|---|---|---|---|---|---|---|---|---|
| Control | | | Nasal Olfactory | | | | | |
| | Onset | | Mucosa | | | Human Embryonic | | |
| | of | | | Start of | | Spinal Cord | | |
| | move- | | | move- | | | Onset of | |
| Rat | ment | BBB | Rat | ment | BBB | Rat | movement | BBB |
| No. | (days) | scale | No. | (days) | scale | No. | (days) | scale |
| 1 | — | 0 | 1 | 15 | 10 | 1 | 15 | 13 |
| 2 | — | 0 | 2 | — | 0 | 2 | 15 | 6 |
| 3 | — | 0 | 3 | 13 | 13 | 3 | 48 | 10 |
| 4 | — | 0 | 4 | 92 | 3 | 4 | 12 | 3 |
| 5 | — | 0 | 5 | — | 0 | | | |
| 6 | — | 0 | 6 | — | 0 | | | |
| 7 | 30 | 1 | 7 | — | 0 | | | |
| 8 | — | 0 | 8 | — | 0 | | | |

Statistical Analyses of the Three Groups of Animals Graded by the BBB Scale

The BBB scale does not follow a normal distribution (Shapiro-Wilk highly significant for 2 out of 3 groups). A non-parametric test is therefore used to compare the groups. The Kruskal-Wallis Test shows a significant difference between the groups (p=0.0117).

To test which of the group pairs is significantly different, the Duncan multiple comparison test was used on the ranked data (as the raw data is non-normal). The mean rank of human embryonic spinal cord group (16.88) is significantly higher than both the human NOM and control groups (means of 10.5 and 7.31, respectively). No significant difference emerged when comparing the NOM and control groups.

Electrophysiological Measurements

Spinal cord conductivities were measured immediately after spinal cord transection and again three months later in two groups-transection alone, or transection plus implantation of composite NOM implants. In 2 out of the 3 NOM implanted-rats exhibiting legs movement, SSEPs were elicited (FIG. 9 section B). No SSEP response was elicited in the 8 rats of the control group, nor in the 5 rats of the NOM implanted group that did not show leg movement (FIG. 9 section A).

MRI Analysis

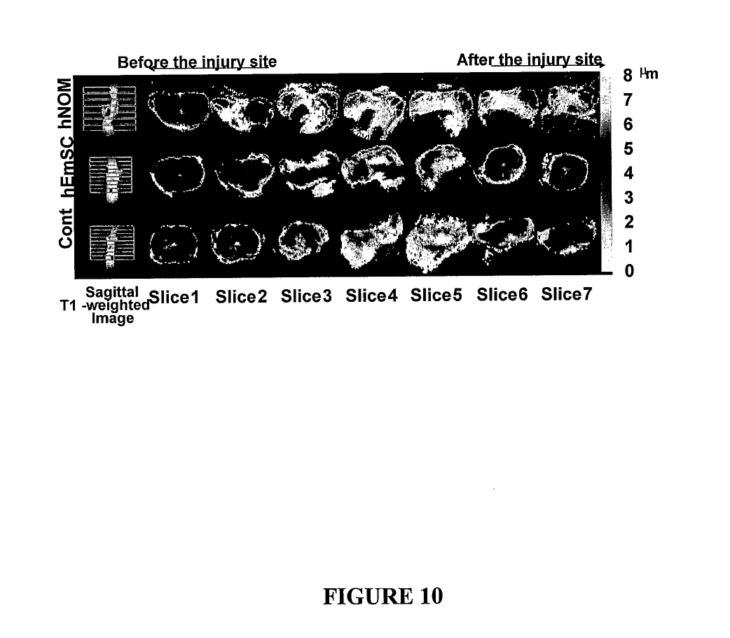

Representatives of the treatment groups were subjected to magnetic resonance imaging (MRI) analysis, which provided information on the state of the spinal cord tissue at the injury site. Magnetic resonance (MR) q-space displacement maps (Assaf Y. and Cohen Y. 2000. Magn Reson Med 43:191-9; Assaf Y, et al., 2000. Magn Reson Med 44:713-22) which were computed for three different spinal cords, revealed that fiber-like tissue with an amount of water-restricted diffusion was present only in the treated spinal cords and not in the controls (FIG. 10). Moreover, comparison of slices numbers 3-5, which are of the implantation sites, show small areas in which the mean displacement is less than 4 μm (value consistent with normal white matter). Such areas are present only in slices of implanted rats, but not in the controls (FIG. 10).

Histological Analysis

The dominant histological picture of the reparative tissue in the area of the excised cord tissue was the presence of fibrotic scar tissue, composed of glial cells and fibroblasts, together with the formation of new blood vessels. No inflammatory reaction was observed either in the histology of rats implanted with human cells or in the control rats implanted with gel alone.

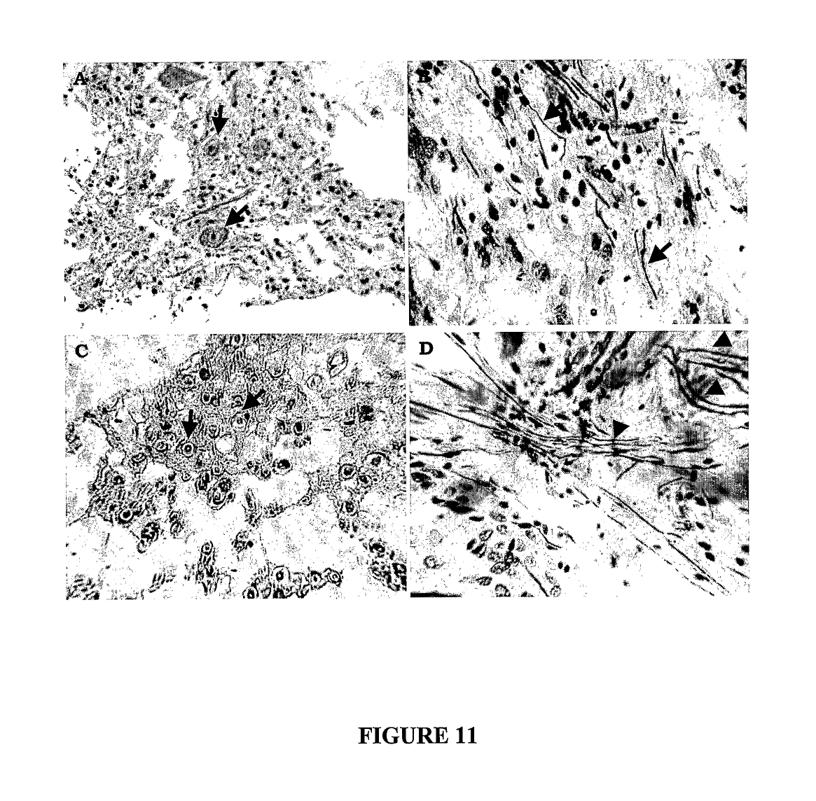

In rats implanted with either human NOM or with embryonic spinal cord the H&E stained sections showed some areas of neurokeratin (shrunk axons surrounded by an empty space, residual of the myelin sheath that had been dissolved by the alcohol treatment). Furthermore, in one rat (that was walking on the right leg after NOM implantation) a number of large neuronal perikarya were observed in the implanted area (FIG. 11, sections A, C). In silver stained sections of both composite implants several nerve fibers could be seen crossing the reparative tissue (FIG. 11, sections B, D)

### Example 6

### Implantation of Cultured Autologous Adult NOM for the Treatment of Traumatic Spinal Cord Injury in Dogs

The dog model for chronic spinal cord injury (cSCI) is a unique model for human cSCI. Dog and human lives are intertwined in many respects. Dogs live with humans in the same place and engage in similar activities. Many of the cSCI that occur in humans occur similarly in dogs (car accidents, gun shots, falls, disc extrusion etc), thus SCI is accidental and not induced like that of laboratory animals. Moreover, since human and dog spinal cords are similar in size they undergo similar surgical procedures.

Paralyzed dogs (From the Veterinary Clinic of Via Emilia, Sordio (LO) and the Department of Veterinary Science-Clinical Section, University of Camerino, Italy) were treated by open surgical procedure which included transplanting a composite implant into the injured spinal cord after removal of the scar. (The treatment was performed with the consent of the dog's owners and the Italian Ministry of health). The composite implant was as described in the rat study herein above, with the exception that the NOM biopsies were taken by rhinoscopy from the central deep turbinate of the dog's nose.

The composite implant was inserted into the gap formed after removal of the persisted scar at the spinal cord injured site. An additional 0.5 ml of HA-LN-Gel containing NOM cells was infiltrated under the dura membrane using 22-gage catheter. The dura was closed using 6/0 absorbable monofilament sutures (Ethilon or b/a dural graft matrix (DuraGen). A low power Helium-Neon laser (632 nm) irradiation was used directly on the implant for 10 minutes at a distance of 2 cm. After the surgical procedure, the dogs received a daily special care for at least 6 months. The treatment included daily physiotherapy, involving swimming, electrical stimulation, ozone and laser therapy, muscle massage and magnetotherapy.

By the end of six months after implantation, all dogs improved and were in a better situation than before the implantation. Although no one of the implanted dogs was yet capable of continues regular walking, two dogs were capable to gain a normal gait position, stood up without any help and walked a few steps before loosing equilibrium. One dog was capable of walking on a treadmill with help, and swimming using his back legs with no help. Another treated dog was able to swim with no help, and additional dog was capable to maintain upright posture, once positioned with help, for few seconds. Furthermore, all dogs retained control on the bladder activity 3-4 months after implantation. Prior to implantation, no leg movement or bladder control could be observed.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

It should be understood that the detailed description and specific examples, while indicating preferred embodiments of the invention, are given by way of illustration only, since various changes and modifications within the spirit and scope of the invention will become apparent to those skilled in the art from this detailed description.

1-73. (canceled)

74. A composition for expanding stem cells, comprising a population of stem cells cultured in or on a biocompatible matrix comprising hyaluronic acid and laminin cross-linked to form a combined gel, further comprising nutrient culture medium, suitable for supporting the growth of stem cells wherein at least the majority of the cells are in their undifferentiated state.

75. The composition according to claim 74, wherein the composition is devoid of a feeder layer or conditioned medium.

76. The composition according to claim 74, wherein the stem cells are selected from stem cells of human origin and stem cells of non-human mammalian origin, and wherein the stem cells are selected from the group consisting of pluripotent embryonic stem cells, pluripotent adult stem cells, partially committed progenitor cells or combinations thereof.

77. The composition according to claim 76, wherein the partially committed progenitor cells are neural progenitor cells selected from spinal cord cells and nasal olfactory mucosa (NOM) cells.

78. The composition according to claim 74, wherein the nutrient culture medium is selected from a serum-free medium and a medium containing serum, wherein the serum is selected from the group consisting of autologous serum and non-autologous serum.

79. The composition according to claim 74, wherein the nutrient culture medium further comprises an agent supporting the growth of the cells in an undifferentiated state, selected from the group consisting of basic fibroblast growth factor (bFGF), epidermal growth factor (EGF), members of the interleukin 6 family (IL-6) and leukemia inhibitory factor (LIF).

80. The composition according to claim 74, wherein the population of stem cells cultured in or on said biocompatible matrix forms a monolayer or an embryoid body structure in the culture.

81. The composition according to claim 74, wherein the population of stem cells cultured in or on said biocompatible matrix substantially maintain their undifferentiated state throughout at least one duplication of the cell culture.

82. The composition according to claim 74, wherein the population of stem cells cultured in or on said biocompatible matrix are genetically modified.

83. A composition comprising a population of cells cultured in or on a biocompatible matrix comprising hyaluronic acid and laminin cross-linked to form a combined gel, further comprising nutrient culture medium, wherein the cell population is derived from stem cells, and wherein at least part of the cells are differentiated to a desired cell type.

84. The composition according to claim 83, wherein the composition is devoid of a feeder layer or conditioned medium.

85. The composition according to claim 83, wherein the cells are selected from cells of human origin and cells of non-human mammalian origin, and wherein the stem cells are selected from the group consisting of pluripotent embryonic stem cells, pluripotent adult stem cells, partially committed progenitor cells or combinations thereof.

86. The composition according to claim 85, wherein the partially committed progenitor cells are neural progenitor cells selected from spinal cord cells and nasal olfactory mucosa (NOM) cells.

87. The composition according to claim 83, wherein the differentiated cells are neural cells.

88. The composition according to claim 84, wherein the nutrient culture medium is selected from a serum-free medium and a medium containing serum, wherein the serum is selected from the group consisting of autologous serum and non-autologous serum.

89. The composition according to claim 83, wherein the nutrient culture medium further comprises an agent supporting the differentiation of the cells, selected from the group consisting of growth factors; differentiation regulators selected from BDNF, retinoic acid, dopamine and NGF; or combinations thereof.

90. The composition according to claim 83, further comprising synthetic or natural polymers in the form of a plurality of carriers dispersed within the combined gel.

91. The composition according to claim 90, wherein the carriers are positively charged microcarriers.

92. A composite implant comprising cells cultured in or on cross-linked hyaluronic acid-laminin gel further comprising nutrient culture medium and a biocompatible scaffold, wherein the scaffold supports the cultured cells.

93. The composite implant according to claim 92, wherein the cells are selected from undifferentiated stem cells, differentiated cells derived from the stem cells or combinations thereof.

94. The composite implant according to claim 93, wherein the stem cells are selected from pluripotent stem cells and partially committed progenitor cells.

95. The composite implant according to claim 94, wherein the partially committed progenitor cells are NOM cells.

96. The composite implant according to claim 93, wherein the differentiated cells are neural cells.

97. The composite implant according to claim 96, wherein the neural cells differentiated from partially committed NOM cells.

98. The composite implant according to claim 92 wherein the cells are selected from cells of human origin and cells of non-human mammalian origin.

99. The composite implant according to claim 92, wherein the scaffold encloses the cultured cells and the cross-linked hyaluronic acid-laminin gel.

100. The composite implant according to claim 99, wherein the scaffold is selected from tubular form further enclosing nanofibers and a grooved tubular form.

101. The composite implant according to claim 92, wherein the scaffold is positively charged.

102. The composite implant according to claim 92, wherein the scaffold comprises a cohesive biopolymer gel comprising a coprecipitate of at least one fibrillar protein and at least one sulfated polysaccharide.

103. The composite implant according to claim 102, wherein the cohesive biopolymer gel comprises of coprecipitate of dextran sulfate and gelatin.

104. The composite implant according to claim 103, wherein the scaffold is sutured without damage to the overall structure.

105. A method for expanding stem cells, comprising: (a) providing a population of stem cells; and (b) culturing the population of stem cells in or on a composition comprising biocompatible matrix comprising hyaluronic acid and laminin cross-linked to form a combined gel, further comprising nutrient culture medium, under conditions wherein the cultured cells are proliferating while maintaining their undifferentiated state.

106. The method according to claim 105, wherein the composition is devoid of a feeder layer or conditioned medium.

107. The method according to claim 105, wherein the population of the stem cells is obtained from a pre-culture grown on a feeder layer in a medium selected from a serum containing culture medium and a serum free culture medium.

108. The method according to claim 105, wherein the stem cells are selected from cells of human origin and cells of non-human mammalian origin, further wherein the stem cells are selected from the group consisting of pluripotent embryonic stem cells, pluripotent adult stem cells, partially committed progenitor cells or combinations thereof.

109. The method according to claim 105, wherein the nutrient culture medium further comprises serum.

110. The method according to claim 105, wherein the nutrient culture medium comprises an agent supporting the growth of the cells in an undifferentiated pluripotent state, selected from the group consisting of basic fibroblast growth factor (bFGF), epidermal Growth Factor (EGF), members of the interleukin 6 family (IL-6) and leukemia inhibitory factor (LIF).

111. The method according to claim 110, wherein the stem cells form a monolayer or an embryoid body structure in the culture.

112. The method according to claim 105, wherein the stem cells maintain their substantially undifferentiated state throughout at least one duplication of the cell culture.

113. The method according to claim 105, further comprising a step of transforming the population of the stem cell with a suitable vector comprising an exogene, wherein the vector is selected from the group consisting of a viral vector, a plasmid vector and a non-viral system.

114. The method according to claim 114, wherein transformation is performed prior to culturing the stem cell population in or on the hyaluronic acid and laminin combined gel or before re-seeding isolated cells during culture passages.

115. A method for differentiating stem cells, comprising: (a) providing a population of stem cells; and (b) culturing the population of stem cells in or on a composition comprising biocompatible matrix comprising hyaluronic acid and laminin cross-linked to form a combined gel, further comprising nutrient culture medium, under conditions wherein at least part of the stem cells differentiate to a desired cell type.

116. The method according to claim 115, wherein the composition is devoid of a feeder layer or conditioned medium.

117. The method according to claim 115, further comprising culturing the cells in a suspension comprising a plurality of microcarriers before culturing said cells in or on the hyaluronic acid and laminin combined gel.

118. The method according to claim 117, wherein the cells are cultured alternately as stationary cultures in or on the hyaluronic acid and laminin combined gel and subsequently in the solution comprising the plurality of microcarriers, wherein the final stage is the stationary culture in or on said hyaluronic acid and laminin combined gel.

119. The method according to claim 115, wherein the stem cells are selected from cells of human origin and cells of non-human mammalian origin, further wherein the stem cells are selected from the group consisting of pluripotent embryonic stem cells, pluripotent adult stem cells, partially committed progenitor cells or combinations thereof.

120. The method according to claim 115, wherein the composition further comprises serum free medium.

121. The method according to claim 115, wherein the culture medium comprises an agent supporting differentiation of the cells, selected from the group consisting of growth factors; differentiation regulators selected from BDNF, retinoic acid, dopamin, and NGF; or any combination thereof.

122. The method according to claim 115, wherein the cells form a monolayer in the culture.

123. A method for transplanting cells to an individual in need thereof, comprising transplanting a composite implant comprising the cells cultured in or on cross-linked hyaluronic acid-laminin gel, and a biocompatible scaffold, wherein the scaffold supports said cultured cell.

124. The method according to claim 123, wherein the cells are transplanted into an injured site of a spinal cord tissue.

125. The method according to claim 124, further comprising covering the site of the injured tissue with a thin biodegradable membrane.

126. The method according to claim 125, wherein the membrane comprises a coprecipitate of dextran sulfate and gelatin.

127. The method according to claim 125, wherein the membrane is attached to the injured site by interstitial sutures.

128. The method according to claim 123, wherein the cells are selected from undifferentiated stem cells, differentiated cells derived from the stem cells or combinations thereof.

129. The method according to claim 128, wherein the cells are selected from pluripotent stem cells and partially committed progenitor cells.

130. The method according to claim 129, wherein the partially committed progenitor cells are adult NOM cells.

131. The method according to claim 128, wherein the differentiated cells are neural cells.

132. The method according to claim 131, wherein the neural cells differentiated from partially committed NOM cells.

133. The method according to claim 123, wherein the scaffold comprises a cohesive biopolymer gel comprising a coprecipitate of at least one fibrillar protein and at least one sulfated polysaccharide.

134. The method according to claim 133, wherein the cohesive biopolymer gel comprises a coprecipitate of dextran sulfate and gelatin.
